(19) United States
(12) Patent Application Publication
(10) Pub. No.: US 2024/0024159 A1
(43) Pub. Date: Jan. 25, 2024

(54) POSTERIOR CHAMBER PLACEMENT OF SUSTAINED RELEASE IMPLANT

(71) Applicant: Allergan, Inc., North Chicago, IL (US)

(72) Inventors: Michael Robinson, Huntington Beach, CA (US); Alazar Ghebremeskel, Irvine, CA (US); Zoran Novakovic, Irvine, CA (US); David Aubuchon, Mission Viejo, CA (US); John Vanden Dries, San Clemente, CA (US)

(21) Appl. No.: 18/004,755

(22) PCT Filed: Jul. 9, 2021
(86) PCT No.: PCT/US2021/041189

§ 371 Date: Jan. 9, 2023

Related U.S. Application Data
(60) Provisional application No. 63/219,440, filed on Jul. 8, 2021.
(60) Provisional application No. 63/050,452, filed on Jul. 10, 2020.

(51) Int. Cl. A61F 9/007; A61F 9/00; A61K 47/38; A61K 31/5575

(52) U.S. Cl. CPC A61F 9/00781; A61F 9/0017; A61K 47/38; A61K 31/5575; A61F 2250/0068

(57) ABSTRACT

A system for reducing intraocular pressure of a patient in need including a delivery device and an intraocular implant positioned within the lumen of the cannula proximal to a retention plug. The delivery device has a housing sized to be held by an operator and an actuator. The delivery device has a cannula defining a lumen and having a proximal end coupled to the housing. The cannula extends along longitudinal axis from the proximal end to a distal end, the distal end of the cannula having rounded inner and outer edges defining a blunt, non-beveled distal opening from the lumen. The retention plug is attached to the cannula spanning the lumen near the distal opening. Related methods, implants and delivery tools are provided.

This application claims the benefit of priority to U.S. Provisional Application Ser. No. 63/050,452, filed Jul. 10, 2020, and U.S. Provisional Application Ser. No. 63/219,440, filed Jul. 8, 2021. The entire contents of these applications are incorporated by reference in their entirety.

### BACKGROUND

Glaucoma is generally a progressive disease of the eye characterized by progressive optic neuropathy with associated visual field loss. Glaucoma may be further associated with increased intraocular pressure. Based on its etiology, glaucoma has been classified as primary or secondary. Primary glaucoma in adults may be either open-angle glaucoma or acute or chronic angle-closure glaucoma. Secondary glaucoma results from pre-existing ocular diseases such as uveitis, intraocular tumor or an enlarged cataract.

The underlying causes of primary glaucoma are not yet known. Risk factors include high or elevated intraocular pressure, advanced age, and family history. Increased or elevated intraocular pressure is due to the obstruction of aqueous humor outflow. In primary open-angle glaucoma, the anterior chamber and its anatomic structures appear normal, but drainage of the aqueous humor is impeded. In acute or chronic angle-closure glaucoma, the anterior chamber is shallow, the filtration angle is narrowed, and the iris may obstruct the trabecular meshwork at the entrance of the canal of Schlemm. Dilation of the pupil may push the root of the iris forward against the angle, and may produce pupillary block and thus precipitate an acute attack. Eyes with narrow anterior chamber angles are predisposed to acute angle-closure glaucoma attacks of various degrees of severity.

Secondary glaucoma is caused by any interference with the flow of aqueous humor from the posterior chamber into the anterior chamber and subsequently, into the canal of Schlemm. Inflammatory disease of the anterior segment may prevent aqueous escape by causing complete posterior synechiea in iris bombe and may obstruct movement of aqueous humor through the pupil leading to elevated intraocular pressure. Other common causes are intraocular tumors, enlarged cataracts, central retinal vein occlusion, trauma to the eye, operative procedures and intraocular hemorrhage. Considering all types together, glaucoma occurs in about 2% of all persons over the age of 40 and may be asymptomatic for years before progressing to noticeable peripheral visual loss followed by central vision loss.

Glaucoma is considered to potentially be both an anterior and posterior ocular condition because a clinical goal of glaucoma treatment can be to not only reduce elevated intraocular pressure because of obstructed aqueous humor outflow from the anterior chamber, but to also prevent the loss of, or reduce the occurrence of loss of, vision due to damage to or loss of retinal cells or optic nerve cells (i.e., ganglion cells) in the posterior of the eye (i.e. neuroprotection). Clinical trials have shown that reducing IOP can help retard the progression of glaucoma and consistent IOP reduction is associated with reduced risks of developing and progressing optic nerve damage.

Patient non-adherence to topical therapy is one of the major challenges to preventing vision loss due to glaucoma. Patients that take no medication are at the highest risk of vision loss from glaucoma; however, patients that intermittently take their medications are also at risk since IOP fluctuation has also been identified as possible risk factor for progression in some patients.

### SUMMARY

In light of the above, new drug delivery devices, systems, and methods would be beneficial, particularly for delivering therapeutic agents to the posterior chamber of the eye. It would be particularly advantageous to provide improved reductions in IOP while mitigating risks of corneal damage.

In an aspect, provided is a system for reducing the intraocular pressure of a patient in need. The system includes a delivery device having a housing sized to be held by an operator and an actuator. The delivery device includes a cannula defining a lumen and having a proximal end coupled to the housing. The cannula extends along a longitudinal axis from the proximal end to a distal end. The distal end of the cannula has rounded inner and outer edges defining a blunt, non-beveled distal opening from the lumen. A retention plug is adhered to the cannula spanning the lumen near the distal opening. An intraocular implant is positioned within the lumen of the cannula proximal to the retention plug.

The cannula can have an exposed working length between the proximal end and the distal end that is between about 12 mm and about 18 mm. The cannula can have an outer dimension sized to extend through a self-sealing corneal incision or puncture. The cannula can be no larger than about 28 gauge. The cannula can have a wall thickness no greater than about 50 μm. An outside surface of the cannula can be siliconized and the lumen of the cannula can be substantially non-siliconized. The retention plug prevents inadvertent release of the implant from the lumen prior to actuation of the device. The retention plug can be formed from a retainer solution of hydroxypropyl methylcellulose (HPMC). The retainer solution can have a viscosity between 6,000 cP and 13,000 cP. The retainer solution can have a concentration greater than about 2.5% and less than about 4%, and preferably about 3%. The retainer solution can be dispensed within the lumen of the cannula as a dispensed mass of greater than 100 μg and less than 300 μg. The retainer solution can be a 3% F4M-HPMC in water having an apparent viscosity of about 8,640 cP to about 12,760 cP and dispensed into the cannula as a dispensed mass between 125 μg-200 μg, the cannula being 28 gauge.

The implant can have a length of no more than 3.0 mm and a maximum width of no more than about 0.5 mm. The intraocular implant can include bimatoprost or a salt thereof present in an amount of about 20% by weight of the implant and a biodegradable polymer matrix comprising at least one biodegradable polymer. The intraocular implant can be DURYSTA™. The intraocular implant can be DURYSTA™ in either a 6 μg, 10 μg, 15 μg, or 20 μg dosage.

The actuator on the housing can move a push rod through the lumen of the cannula to push the implant out from the lumen via a linkage. The actuator can be coupled to the push rod through the linkage, the push rod being movable along the longitudinal axis as the linkage is gradually flattened as the actuator is depressed. The push rod can have a length relative to a length of the cannula sufficient for a distal end of the push rod to advance past the distal end of the cannula upon deployment of the implant using the actuator.

In an interrelated aspect, provided is a system for reducing intraocular pressure of a patient in need that includes a delivery device having a housing sized to be held by an operator and an actuator. The delivery device includes a push rod linked to the actuator and a cannula having a tubular wall extending along a longitudinal axis between a proximal end coupled to the housing and a distal end. The tubular wall defines a lumen sized to slidably receive the push rod. The distal end of the cannula has rounded inner and outer edges defining a blunt, non-beveled distal opening into the lumen. The tubular wall between the proximal end and the distal end is about 12 mm and about 18 mm long. The tubular wall is siliconized on its external surface and the lumen is non-siliconized. A retention plug is contained within and spanning the lumen near the distal opening. The retention plug is formed from a dispensed mass of 3% hydroxypropyl methylcellulose (HPMC) retainer solution. An intraocular implant is positioned within the lumen of the cannula proximal to the retention plug and distal to the push rod. The implant includes 20% by weight bimatoprost or a salt thereof and a biodegradable polymer matrix having at least one biodegradable polymer.

In an interrelated aspect, provided is a method for improving the efficacy of a bimatoprost-containing intraocular implant in reducing intraocular pressure of a patient in need thereof. The method includes positioning a single bimatoprost-containing intraocular implant into a posterior chamber of an eye of the patient. The single bimatoprost-containing intraocular implant causes a greater reduction in intraocular pressure compared to an equivalent bimatoprost-containing intraocular implant positioned into an anterior chamber of the eye of the patient closer to a trabecular meshwork of the eye. The bimatoprost-containing intraocular implant can include 6, 10, 15, or 20 μg of bimatoprost or a salt thereof that elutes over a period of up to about 6 months. The implant can be effective to reduce the intraocular pressure of the patient over a period of time between about 12 months and about 24 months or longer. The method can further include advancing a blunt-tipped cannula having a lumen containing the implant through the anterior chamber over at least a portion of the pupil and under at least a portion of the iris; pushing the implant through the lumen of the cannula past a retention plug attached to the cannula so as to span the lumen and out a distal opening defined by rounded inner and outer edges of the cannula; and releasing the implant within a region of the posterior chamber of the eye behind the iris. In some variations, one or more of the following can optionally be included in any feasible combination in the above compositions, methods, devices, and systems. More details of compositions, methods, devices, and systems are set forth in the accompanying drawings and the description below. Other features and advantages will be apparent from the description and drawings.

### BRIEF DESCRIPTION OF THE DRAWINGS

These and other aspects will now be described in detail with reference to the following drawings. Generally speaking, the figures are not to scale in absolute terms or comparatively, but are intended to be illustrative. Also, relative placement of features and elements may be modified for the purpose of illustrative clarity.

FIG. 1A shows a cross-section of the mammalian eye.

FIG. 1B shows the mammalian eye visualized by a high-definition optical coherence tomography system (HD-OCT).

FIG. 1C shows the canonical schematic of the aqueous humor secreted from the posterior chamber of the eye by the ciliary body through the pupil and into the anterior chamber.

FIG. 1D shows the mammalian eye with an implant positioned within the posterior chamber visualized by a HD-OCT system.

FIG. 1E shows forming an incision in a cornea using a sharpened tool,

FIG. 1F shows an applicator having an implant within the cannula being inserted through the cornea via the incision shown in FIG. 1E.

FIG. 1G shows the cannula being positioned through the pupil and behind the iris to deploy the implant within the posterior chamber.

FIG. 1H shows the implant deployed from the cannula of the applicator and positioned within the ciliary sulcus.

FIG. 2A is a perspective view of an implant delivery apparatus.

FIG. 2B is a partially exploded side view of the housing, linkage, and actuating lever of an implant delivery apparatus.

FIG. 2C is a cross-sectional view of an implant delivery apparatus;

FIG. 2D is an enlarged side view of a nose cone and cannula of the implant delivery apparatus of FIG. 2A.

FIG. 2E is an enlarged view of the cannula of FIG. 2D.

FIG. 2F is a cross-sectional view of the cannula of FIG. 2E showing the presence of the retention plug.

FIG. 3 shows an enlarged perspective view of the linkage of the implant delivery apparatus shown in FIG. 2C.

FIG. 4A shows an enlarged perspective view of the actuating lever of the implant delivery apparatus shown in FIG. 2C.

FIG. 4B shows an enlarged perspective view of the actuating lever of FIG. 4A.

FIG. 5 shows a cross-sectional, partial view of a safety tab engaged with a linkage.

FIG. 6 shows mean percentage change in IOP from baseline in beagle dogs over 3 months after placement of different doses of anterior chamber implants.

FIG. 7 shows IOP reduction with posterior chamber implant containing bimatoprost (solid bar) compared to control (hatched bar) after 1 week.

FIG. 8 shows central corneal endothelial cell counts for implant containing bimatoprost (solid bar) compared to control (hatched bar) are stable after dosing with a posterior chamber implant after 1 week.

It should be appreciated that the drawings are for example only and are not meant to be to scale. It is to be understood that devices described herein may include features not necessarily depicted in each figure.

### DETAILED DESCRIPTION

Described herein is a new and improved method for delivering biodegradable intraocular implants into the posterior chamber of the eye. The method provides superior benefits over administering such implants into the anterior chamber, including enhanced therapeutic effects, decreased risk of vision loss, decreased risk of causing damage to the corneal endothelium, and/or decreased risk of corneal endothelial cell density loss. Preferably, the method may be used to deliver biodegradable implants that provide for the extended release of prostamides such as bimatoprost in an amount that is effective for treating an ocular condition, particularly glaucoma and ocular hypertension, and conditions associated with glaucoma such as elevated intraocular pressure.

Also described herein is a device for administering biodegradable intraocular implants into the posterior chamber of the eye. The device is designed specifically to deliver such implants into the posterior chamber, including the ciliary sulcus and/or atop or adjacent to the ciliary zonules, using a blunt-tipped cannula with rounded edges having an extended cannula length and an optionally siliconized outside cannula surface. The cannula includes a plug for retaining the implant within the lumen prior to ejection into the posterior chamber. The device permits reliable and safe delivery of each implant into the posterior chamber compared to other insertion devices while providing a significantly decreased risk of accidental trauma to the iris, lens capsule and corneal surfaces.

The implants are sized and configured for placement in the posterior chamber of the eye where the implant can deliver therapeutics such as prostamide or other therapeutic useful for treating glaucoma, to the tissues regulating the production and outflow of aqueous humor. The intraocular implants described here are designed to provide a patient with intraocular pressure-lowering levels of drug for a sustained period lasting for 2 months or more.

Prostamides are potent ocular hypotensive agents useful in the treatment of a number of various ocular hypertensive conditions such as glaucoma, elevated intraocular pressure, and other ocular hypertensive episodes, including post-surgical and post-laser ocular hypertensive episodes. They belong to the family of prostaglandin F_{2α} C-1 amides as discussed in more detail in U.S. Pat. No. 9,492,316, which is incorporated by reference herein.

Commercially available prostamides include bimatoprost, which exhibits no meaningful interaction with prostaglandin (PG) sensitive receptors. Nevertheless, bimatoprost is a potent ocular anti-hypertensive agent and is highly effective for reducing elevated intraocular pressure in patients with open angle glaucoma or ocular hypertension. Bimatoprost is typically prescribed for use by patients in the form of an ophthalmic solution known by the tradename LUMIGAN®. In the usual course of therapy, Patients apply one drop of LUMIGAN® solution once daily to the surface of the affected eye(s) to reduce elevated intraocular pressure.

Bimatoprost is believed to decrease intraocular pressure (IOP) by increasing aqueous humor outflow, either by enhancing the pressure-sensitive (presumed trabecular) outflow pathway or by increasing the pressure-insensitive (uveoscleral) outflow without significantly affecting the aqueous production rate (Lim et al. Ophthalmology. 2008 May; 115(5): 790-795.e4.).

Implants exist that are sized to fit within the anterior chamber angle of the eye to deliver directly to these uveoscleral outflow pathways (see, U.S. Pat. No. 9,492,316 and WO 2019/094652, which are each incorporated by reference herein). Release of a drug from an erodible polymer is the consequence of several mechanisms or combinations of mechanisms. Some of these mechanisms include desorption from the implant surface, dissolution, diffusion through porous channels of the hydrated polymer and erosion. The release of the therapeutic agent from the intraocular implant comprising a biodegradable polymer matrix may include an initial burst of release followed by a gradual increase in the amount of the therapeutic agent released, or the release may include an initial delay in release of the therapeutic agent followed by an increase in release. Fick's Second Law of Diffusion explains the behavior of non-steady state diffusion, i.e. diffusion that changes with time. Fick's Second Law is useful to predict the diffusion of a drug and the optimum implantation site of an implant containing the drug as discussed in U.S. Pat. No. 8,571,802, which is incorporated by reference herein. The drug concentrations in the ocular tissues closest to the implant would be highest and more therapeutic compared with drug concentrations further away from the implant. Thus, existing diffusion-based implants for enhancing uveoscleral outflow of aqueous from the anterior chamber were preferably positioned within the anterior chamber because based on Fick's Second Law the ideal implant position is nearest the outflow pathway (i.e., the trabecular meshwork) being treated. Conventionally, Fick's Second Law provided a basis for why implants for treating glaucoma are positioned more anteriorly (i.e. within the anterior chamber) and implants for treating macular diseases are positioned more posteriorly (i.e., within the vitreous). However, positioning implants into the posterior chamber has a number of advantages over the traditional anterior placement as described elsewhere herein.

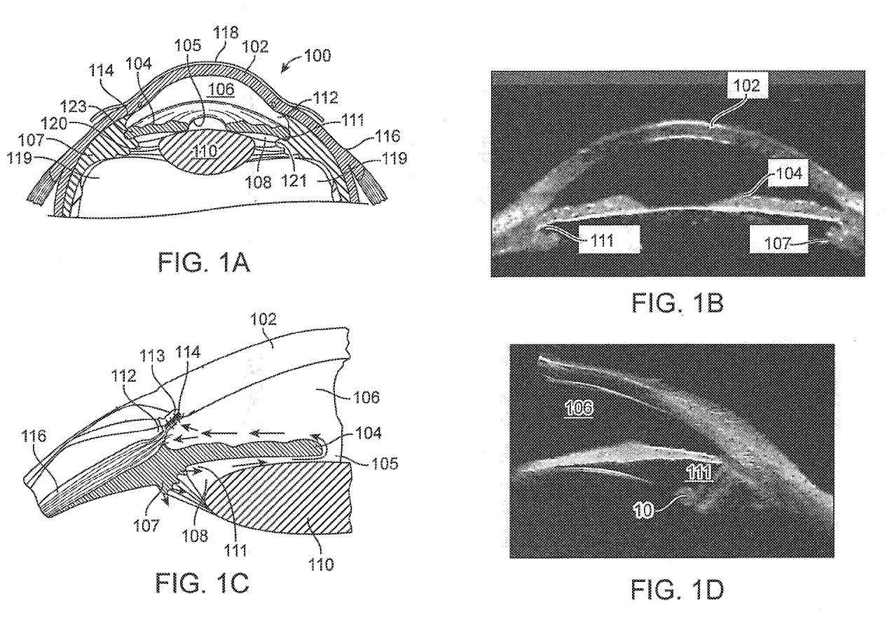

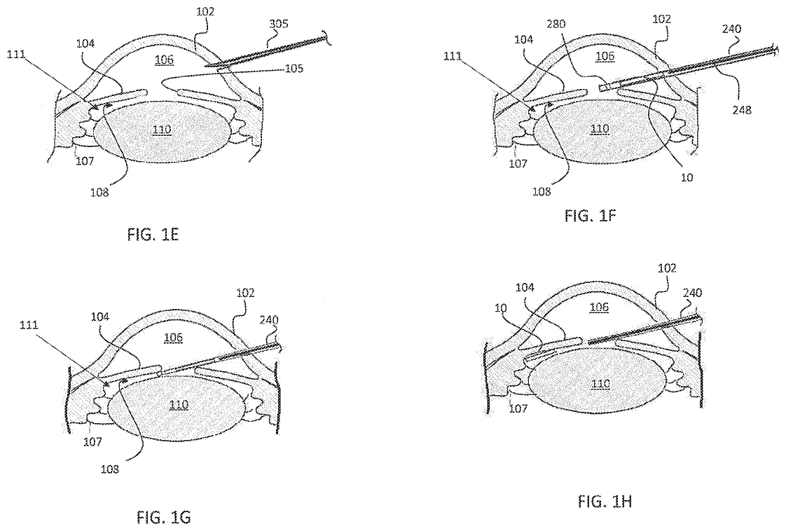

FIGS. 1A-1D show cross-sections of an eye 100. Particular regions of the eye 100 include the cornea 102, iris 104, ciliary body 107, and ciliary sulcus 111. The cornea 102 and iris 104 surround the anterior chamber 106. Within the anterior chamber is the anterior chamber angle 112 and trabecular meshwork 114. Also shown are the corneal epithelium 118, sclera 116, vitreous 119, ciliary zonules 120, and ciliary process 121. The vitreous chamber of the eye is the rear two-thirds of the eyeball (behind the lens 110), and includes the vitreous 119, the retina, and the optic nerve. The posterior chamber 108 and lens 110 are behind the iris 104.

The ciliary body 107 continuously forms aqueous humor in the posterior chamber 108 by secretion from the blood vessels. The aqueous humor flows around the lens 110 and iris 104 into the anterior chamber 106 and exits the eye 10 through the trabecular meshwork 114 situated at the iridocorneal angle 112 (see arrows of FIG. 1C). Some of the aqueous humor filters through the trabecular meshwork 114 near the iris root into Schlemm's canal 113, a small channel that drains into the ocular veins. A smaller portion rejoins the venous circulation after passing through the ciliary body 107 and eventually through the sclera 116 (the uveoscleral route).

The posterior chamber 108 refers to the narrow, fluid-filled space inside the eye 100 that is posterior to the anterior chamber 106 and anterior to the vitreous chamber 119. The posterior chamber 108 is bordered by the anterior zonules 120, anterior lens capsule, anterior ciliary body 107, and the back of the iris 104. The posterior chamber 108 includes the space posterior to the peripheral part of the iris 104 and anterior to the zonules 120, and includes the ciliary sulcus 111. The volume of the ciliary sulcus 111 can vary from patient-to-patient, but generally includes the space of the posterior chamber 108 between the posterior side of the iris 104 and the anterior side of the ciliary body 107. The deepest parts of the ciliary sulcus 111 are near the juncture of the posterior surface of the iris root 123 and the anterior surface of the ciliary body 107 and the shallow region of the ciliary sulcus 111 extending away from this deep location towards the tip of the ciliary processes 121.

Anterior chamber implants are typically inserted through the cornea 102 using a sharpened, beveled cannula and ejected within the anterior chamber 106 such as between the iris 104 and the innermost corneal surface, the corneal endothelium. The anterior chamber implants tend to settle inferiorly into the angle of the anterior chamber 106 (the junction between the anterior surface of the iris 104 and the back surface of the cornea 102, also called the iridocorneal angle 112 (see FIG. 1A). The external surface of the cornea 102 is covered by the corneal epithelium 118 and the internal surface of the cornea 102 is covered by a thin, delicate layer of endothelial cells. Anterior chamber implants are preferentially positioned in contact with the trabecular meshwork 114 and the anterior ciliary muscle tip in the iridocorneal angle 112 of the anterior chamber 106 since those are the principal aqueous outflow pathways to lower IOP. However, the implant positioned in this location has the potential for injuring the corneal endothelium and obstructing vision. Corneal endothelial cell touch can contribute to corneal edema that leads to cloudiness of normally transparent cornea and may result in vision loss if it extends to the central cornea. Placement of implants within the posterior chamber overcomes these limitations. In addition, placement of implants into the posterior chamber also provides the attending physician with the further benefit of providing a viable alternative mode of delivering therapeutics useful for treating glaucoma for those patients that may be particularly sensitive to such therapeutics being placed in the more traditional anterior chamber location—potentially permitting multiple implants to be delivered either concurrently or consecutively—due to the enhanced safety profile of delivering an implant within the posterior chamber.

Implant delivery apparatus that ejects an intracameral implant into the anterior chamber must deliver the implant with a force that is sufficient to drive the implant away from the tip of the needle so that it does not adhere to the needle, which, as the needle is withdrawn from the eye, can damage endothelial cells causing corneal edema and inflammation. On the other hand, implants that are ejected too forcefully may strike the iris or other side of the anterior chamber, which can cause hemorrhages and also damage the endothelium. Still further, implants positioned in the anterior chamber can also damage the endothelium due to contact between the implant and cornea over the duration of drug delivery.

The implants described herein are positioned within the posterior chamber, including atop the zonules or within the deeper regions of the ciliary sulcus 111, offering additional improvements relative to existing biodegradable intraocular implants positioned within the anterior chamber. FIGS. 1C and 1D are HD-OCT images of an eye. FIG. 1D shows an implant 10 positioned within the posterior chamber. FIGS. 1E-1H show in schematic an example method of deploying an implant 10 within the posterior chamber, such as within the ciliary sulcus 111. Placement of the implants within the posterior chamber of the eye releases a therapeutically effective amount of the bimatoprost providing patients with long-lasting relief from ocular hypertension while also avoiding injury to the corneal endothelium. Surprisingly, the posterior chamber implants are more effective in reducing IOP than equivalent implants placed within the anterior chamber nearer to the aqueous outflow paths. Fick's Second Law would predict that an implant positioned within the posterior chamber 108 that is positioned a distance away from a target outflow pathway (i.e., the trabecular meshwork 114) would be less effective in reducing IOP compared to an implant implanted nearer to the target outflow pathway due to a reduction in drug concentration at the target tissue. For example, an implant positioned within the posterior chamber 108 is a distance of approximately 3 mm to 5 mm further away from the trabecular meshwork 114 than an implant positioned within the anterior chamber 106. As will be discussed in more detail below, the drug concentration in the target tissues calculated using Fick's Second Law is reduced for the posterior chamber implant compared to the anterior chamber implant. However, despite these predictions, administration of the implant into the posterior chamber surprisingly resulted in reducing lower intraocular pressure (IOP) when compared to the IOP after anterior chamber administration of the same implant. The single Bimatoprost-containing intraocular implant caused a greater reduction in IOP compared to an equivalent Bimatoprost-containing intraocular implant positioned into the anterior chamber of the eye closer to the trabecular meshwork of the eye.

### Definitions

The following definitions are included for the purpose of understanding the present subject matter and for constructing the appended patent claims. Abbreviations used herein have their conventional meaning within the chemical and biological arts.

An “intraocular implant” refers to a solid or semi-solid drug delivery system or element that is sized and configured to be placed in an ocular region of the eye, including, for example, the anterior chamber. Other ocular regions of the eye into which an intraocular implant can be placed include the vitreous body, subconjunctival space, and subtenon space. Intraocular implants may be placed in an eye without significantly disrupting vision of the eye. Examples of an intraocular implant include extruded biodegradable filaments, such as a rod-shaped implant produced by a hot-melt extrusion process, comprising a biodegradable polymer matrix and a pharmaceutically active agent, associated with the polymer matrix, and cut to a length suitable for placement in an eye. Intraocular implants are biocompatible with the physiological conditions of an eye and do not cause adverse reactions in the eye. In certain forms of the present invention, an intraocular implant may be configured for placement in the anterior chamber, posterior chamber, subconjunctival space, or vitreous body of the eye. Intraocular implants can be biodegradable and may be configured in the form of a cylindrical or non-cylindrical rod produced by an extrusion process. According to some embodiments, the intraocular implant may comprise an active agent effective for treating a medical condition of the eye.

An “intracameral” implant is an intraocular implant that is sized and configured for placement in the anterior chamber of the eye. The anterior chamber refers to the space inside the eye between the iris and the innermost corneal surface (endothelium). An intracameral implant is also an intraocular implant that can fit into the anterior chamber angle (iridocorneal angle) of the eye without contacting the corneal endothelium and thereby without causing corneal trauma, inflammation, or edema, or iris chaffing. One example of an intracameral implant is a hot-melt extruded, biodegradable, rod-shaped filament comprising or consisting of a biodegradable polymer matrix and an active agent associated with the polymer matrix and cut to a length suitable for placement in the anterior chamber of a mammalian eye (for example, a human eye). A rod-shaped intracameral implant can be 0.5 mm to 3 mm in length and 0.05 mm to 0.5 mm in diameter or maximum width in the case of non-cylindrical rods. An intracameral implant is usually between 20 μg and 150 μg in total weight and can fit into the anterior chamber angle (iridocorneal angle) of the eye without contacting the corneal endothelium and thereby without causing corneal trauma, inflammation, or edema, or iris chaffing. For example, the intracameral implant delivered with the present apparatus into the anterior chamber of a mammalian eye, such as a human eye, can be 0.5 mm to 2.5 mm in length, 0.15 mm to 0.3 mm in diameter, and 20 μg to 120 μg in total weight. The intracameral implant can be DURYSTA™. The intracameral implant is preferably deliverable through a 27 gauge, 28 gauge, 29 gauge, or 30 gauge shaft. The inner diameter of the shaft may vary, depending on whether the shaft is a standard or ultra (or extra) thin-wall needle. The diameter, width, or cross-sectional area of the implant should be receivable in the lumen of the needle so that the implant can slidably translate through the lumen of the needle.

A “posterior chamber” implant is an intraocular implant that is structured, sized, or otherwise configured to be placed in the posterior chamber of an eye. The posterior chamber of the eye refers to the narrow, fluid-filled space inside the eye that is posterior to the anterior chamber and anterior to the vitreous chamber. The posterior chamber includes the space posterior to the peripheral part of the iris and anterior to the zonules of the lens, and includes the ciliary sulcus. A posterior chamber implant may be positioned within a region of the ciliary sulcus, within or around the zonules, the ciliary process, and the ciliary muscle. Posterior chamber implants are preferably no more than about 3 mm in length and no more than about 0.5 mm in diameter or maximum width. A posterior chamber implant is usually less than about 300 μg in total weight and can fit into the ciliary sulcus of the eye. Posterior chamber implants can fit within the ciliary sulcus of the eye without applying a tension on or a significant force against neighboring tissues. The posterior chamber implant resides within the posterior chamber without impacting the natural size or shape of the chamber. The posterior chamber implant preferably does not deform tissues within the posterior chamber significantly, but rather passively resides there in order to deliver drug. The posterior chamber implant can be DURYSTA™. The posterior chamber implant is preferably deliverable through a 27 gauge, 28 gauge, 29 gauge, or 30 gauge shaft. The inner diameter of the shaft may vary, depending upon whether the shaft is a standard or ultra (or extra) thin-wall needle. The diameter, width, or cross-sectional area of the implant should be receivable in the lumen of the shaft so that the implant can slidably translate through the lumen.

“Intraocular pressure” (IOP) refers to the fluid pressure in the eye and is determined by the difference in the rate of aqueous humor secretion and outflow. Approximately 90% of the aqueous humor secreted exits through the trabecular meshwork in the anterior chamber. Resistance to outflow can lead to elevated intraocular pressure. Some populations or patient groups with normal tension (i.e., normotensive) glaucoma may have an IOP of from about 11 to 21 mm Hg. Some patient groups or patients with elevated intraocular pressure or ocular hypertension may have an IOP of greater than 20 or 21 mm Hg, as measured with a tonometer. Implants of the present disclosure are expected to be capable of reducing intraocular pressure in both normotensive and hypertensive glaucoma patients.

The terms “ocular condition” and “medical condition of the eye” are synonymous and used interchangeably herein and refer to a disease, ailment, or condition which affects or involves the eye or one of the parts or regions of the eye, including the anterior or posterior regions of the eye. The eye is the sense organ for sight. Broadly speaking the eye includes the eyeball and the tissues and fluids which constitute the eyeball, the periocular muscles (such as the oblique and rectus muscles) and the portion of the optic nerve which is within or adjacent to the eyeball. Non-limiting examples of a medical condition of the eye (i.e., ocular condition) within the scope of the present disclosure include ocular hypertension (or elevated intraocular pressure), glaucoma, dry eye, and age-related macular degeneration. Glaucoma in a patient may be further classified as open-angle glaucoma or angle-closure glaucoma. In one possible method, the patient receiving a drug-containing implant using an apparatus according to this disclosure may have or be specifically diagnosed with primary open-angle glaucoma. A given patient having open-angle glaucoma may have low, normal, or elevated intraocular pressure. Other forms of glaucoma within the present disclosure include pseudoexfoliative glaucoma, developmental glaucoma, and pigmentary glaucoma.

“Associated with a biodegradable polymer matrix” means mixed with, dissolved and/or dispersed within, encapsulated by, surrounded and/or covered by, or coupled to.

The term “biodegradable,” as in “biodegradable polymer” or “biodegradable implant,” refers to an element, implant, or a polymer or polymers which degrade in vivo, and wherein degradation of the implant, polymer or polymers over time occurs concurrent with or subsequent to release of the therapeutic agent. A biodegradable polymer may be a homopolymer, a copolymer, or a polymer comprising more than two different structural repeating units. The terms biodegradable and bioerodible are equivalent and are used interchangeably herein.

“Active agent,” “drug,” “therapeutic agent,” “therapeutically active agent,” and “pharmaceutically active agent” are used interchangeably herein to refer to the chemical compound, molecule, or substance that produces a therapeutic effect in the patient (human or non-human mammal in need of treatment) to which it is administered and that is effective for treating a medical condition of the eye.

The term “patient” can refer to a human or non-human mammal in need of treatment of a medical condition of the eye.

The term “treat”, “treating”, or “treatment” as used herein, refers to reduction, resolution, or prevention of an ocular condition, ocular injury or damage, or to promote healing of injured or damaged ocular tissue. A treatment is usually effective to reduce at least one sign or symptom of the ocular condition or risk factor associated with an ocular condition.

As used herein, “self-sealing” methods of delivering intraocular implants into the eye refers to methods of introducing implants through a needle and into desired locations of a patient's eye without the need for a suture, or other like closure means, at the needle puncture site. Such “self-sealing” methods do not require that the puncture site (where the needle penetrates the eye) completely seal immediately upon withdrawal of the needle, but rather that any initial leakage is minimum and dissipates in short order such that a surgeon or another equally skilled in the art, in his or her good clinical judgment, would not be compelled to suture or otherwise provide other like closure means to the puncture site. Generally, insertion devices that are no larger than about 25 gauge, about 26 gauge, about 27 gauge, about 28 gauge, about 29 gauge, or about 30 gauge or small, are considered self-sealing in this context. Generally, insertion devices that are larger than 25 gauge are not self-sealing unless their use is accompanied by a therapeutic or other agent, such as a gelling agent or filler, which acts to minimize leakage.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used in the present disclosure, whether in a transitional phrase or in the body of a claim, the terms “comprise(s)” and “comprising” are to be interpreted as having an open-ended meaning. That is, the terms are to be interpreted synonymously with the phrases “having at least” or “including at least.” When used in the context of a process the term “comprising” means that the process includes at least the recited steps, but may include additional steps. When used in the context of a molecule, compound, or composition, the term “comprising” means that the compound or composition includes at least the recited features or components, but may also include additional features or components.

For the purposes of promoting an understanding of the embodiments described herein, reference made to preferred embodiments and specific language are used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. All percentages and ratios used herein, unless otherwise indicated, are by weight.

The term “more” as used in the present disclosure does not include infinite number of possibilities. The term “more” as used in the present disclosure is used as a skilled person in the art would understand in the context in which it is used. For example, more than “36 months” implies, as a skilled artisan would understand, 37 months or the number of months the ocular insert can be or is used by a subject, which is greater than 36 months, without loss of efficacy of the therapeutic agent in the insert. Similarly, for example, more than “24 months” implies, as a skilled artisan would understand, 25 months or the number of months the ocular insert can be or is used by a subject, which is greater than 36 months, without loss of efficacy of the therapeutic agent in the insert

Unless otherwise defined, all technical and scientific terms used herein have the same meaning as commonly understood by one of ordinary skill in the art to which this invention belongs. In the case of conflict, the present specification will control. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

The presently disclosed intraocular implants may be effective in treating an ocular condition in an eye of a patient, including an ocular condition associated with elevated intraocular pressure, and more specifically in reducing at least one sign or symptom of, or risk factor for glaucoma. The method generally comprises placing a biodegradable intraocular implant in an ocular region of the eye(s) of the patient affected by the ocular condition. One embodiment is a method for reducing intraocular pressure in a patient suffering from elevated intraocular pressure, ocular hypertension, or glaucoma, comprising placing a prostamide-containing biodegradable intraocular implant in an eye of the patient to thereby reduce intraocular pressure in the eye. Controlled and sustained administration of a prostamide such as bimatoprost to the eye through the use of one or more of the intraocular prostamide-containing implants described here may improve glaucoma treatment by reducing intraocular pressure in a patient suffering from glaucoma or ocular hypertension for an extended period of time, such as for 4, 5, or 6 months or more following placement of the implant in the eye. Implantation of one or two implants of the present disclosure into an eye of a patient may possibly reduce the diurnal fluctuation in intraocular pressure (IOP) in the eye for about two months or longer as compared to the diurnal fluctuation in an eye treated with once daily topical administration of bimatoprost to an eye.

In some implementations, controlled and sustained administration of bimatoprost to the eye through the use of one or more of the intraocular implants described here occurs over a period of time that is shorter than the IOP-reducing effects occur. For example, the bimatoprost can be administered to the eye from the implants (e.g. implant containing about 6, 10, 15, or 20 ug bimatoprost) for 1, 2, 3, 4, 5, or up to about 6 months whereas the IOP-reducing effects of the bimatoprost exist for at least 12 months, 18 months, 24 months, or more. The IOP-reducing effects of the bimatoprost can last longer than the bimatoprost elutes from the implant.

As described above, the implants comprise or consist of a prostamide and a biodegradable polymer matrix that is formulated to release the prostamide over an extended period of time, such as 60 days or longer. A polyethylene glycol, such as PEG 3350, may optionally be included in the implant. The prostamide may comprise a compound having Formula I.

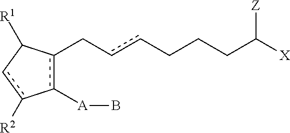

wherein the dashed bonds represent a single or double bond which can be in the cis or trans configuration, A is an alkylene or alkenylene radical having from two to six carbon atoms, which radical may be interrupted by one or more oxide radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is independently selected from the group consisting of hydrogen and a lower alkyl radical having from one to six carbon atoms; Z is ═O; one of R¹ and R² is ═O, OH or a —O(CO)^{R} group, and the other one is —OH or —O(CO)R⁶, or R¹ is ═O and R² is H, wherein R₆ is a saturated or unsaturated acyclic hydrocarbon group having from 1 to about 20 carbon atoms, or —(CH₂)mR₇ wherein m is 0 or an integer of from 1 to 10, and R⁷ is cycloalkyl radical, having from three to seven carbon atoms, or a hydrocarbyl aryl or heteroaryl radical, as defined above.

In a more specific embodiment, the prostamide contained by the implant is bimatoprost, which has the following chemical structure:

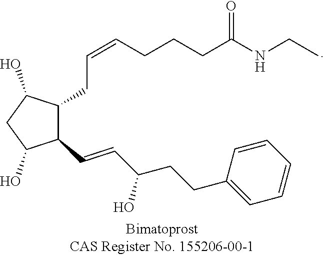 [SMILES: CCNC(=O)CCC/C=C\C[C@@H]1[C@@H](/C=C/[C@@H](O)CCc2ccccc2)[C@H](O)C[C@@H]1O]

In other embodiments, the intraocular implants for delivery into the posterior chamber and/or ciliary sulcus may contain a prostaglandin or prostaglandin analog suitable for treatment of glaucoma as described herein. The prostaglandin analog can include one or more of latanoprost (XALATAN®), bimatoprost (LUUIGAN® or LATISSE®), carboprost, unoprostone, prostamide, travatan, travoprost, or tafluprost, for example, and other therapeutic agents such as prostaglandin precursors, including anti-glaucoma drugs. Anti-glaucoma drugs include beta-blockers, such as timolol, betaxolol, levobetaxolol, and carteolol; miotics, such as pilocarpine; carbonic anhydrase inhibitors, such as brinzolamide and dorzolamide; seretonergics; muscarinics; dopaminergic agonists; and adrenergic agonists, such as apraclonidine and brimonidine.

The intraocular implants are intended to provide a therapeutically effective amount of the prostamide into an ocular region of the eye, preferably the posterior chamber, for 2-4 months or longer, such as 12 months, 18 months, 24 months, or longer. Thus, with a single administration of the implant, a therapeutically effective amount of a prostamide will be made available near the site where it is needed and the therapeutic effect will be maintained for an extended period of time, rather than subjecting the patient to repeated injections or, in the case of self-administered eye drops, the burden of daily dosing.

The implant may be monolithic, i.e. having the active agent (for example bimatoprost) homogenously distributed throughout the polymeric matrix. Alternatively, the active agent may be distributed in a non-homogenous pattern in the polymer matrix. For example, an implant may include a portion that has a greater concentration of the prostamide compound relative to a second portion of the implant.

The implant may be effective for maintaining intraocular pressure in an eye at a reduced level (relative to the intraocular pressure in the eye compared to an implant positioned in the anterior chamber) for at least 2 months, 3 months, 4 months, 5 months, 6 months up to about 24 months and in some cases longer. The percent relative reduction in IOP over baseline in an eye after receiving the implant in the posterior chamber may vary, depending on the size of the implant (and therefore the drug load) and on the patient, but may be at least 10%, 20%, 25%, 30%, 35%, 40%, 45%, 50%, up to about 60% reduction in IOP from baseline. The reduction may be from 10-20%, 20-30%, or 10-50% below baseline IOP (the intraocular pressure in the eye before receiving the implant) and may, in some instances, remain at 20-30% below baseline IOP for at least 2 months, 2-3 months, 4-6 months or longer, and in some instances for up to 24 months or longer after implantation of a single implant.

In general, an intraocular implant can include bimatoprost as the active agent, a biodegradable polymer matrix, and optionally a polyethylene glycol. The bimatoprost (or other prostamide) may be from 5% to 90% by weight of the implant, or from 5% to 30% by weight of the implant, or from 18-22% by weight of the implant, but is preferably 20% by weight of the implant. The biodegradable polymer matrix will generally be a mixture of at least three different biodegradable polymers independently selected from the group consisting of poly(D,L-lactide) (PLA) polymers and poly(D,L-lactide-co-glycolide) (PLGA) polymers. For example, the biodegradable polymer matrix may comprise or consist of first, second, and third biodegradable polymers that differ one from the other by their repeating unit, inherent viscosity, or end-group, or any combination thereof. In some instances, the biodegradable polymer matrix according to the present disclosure may comprise first, second, third, and fourth biodegradable polymers independently selected from the group consisting of poly(D,L-lactide) (PLA) polymers and poly(D,L-lactide-co-glycolide) (PLGA) polymers, wherein the first, second, third, and fourth polymers differ one from the other by their repeating unit, inherent viscosity, or end-group, or any combination thereof. Depending on the chain terminating agent used during the synthesis of the polymer, a PLA or PLGA polymer may have a free carboxylic acid end group or alkyl ester end group, and may be referred to herein as an acid-end or ester-end (or ester-capped) PLA or PLGA polymer, respectively.

One example of an intraocular implant (i.e., drug delivery system) is an extruded biodegradable intraocular implant sized for implantation in the posterior chamber of an eye, the implant comprising or consisting of 18% to 22% (e.g., 20%) by weight (w/w) bimatoprost, 3.5% to 6.5% (e.g., 5%) by weight PEG 3350, 18% to 22% (e.g., 20%) by weight R203S, which is an ester-end poly(D,L-lactide) polymer having an inherent viscosity of 0.25-0.35 dl/g, 13.5% to 16.5% (e.g., 15%) by weight R202H, which is an acid-end poly(D,L-lactide) polymer having an inherent viscosity of 0.16-0.24 dl/g, and 36% to 44% (e.g., 40%) by weight RG752S, which is an ester-end poly(D,L-lactide-co-glycolide) polymer having a D,L-lactide:glycolide molar ratio of about 75:25 and an inherent viscosity of 0.16-0.24 dl/g, wherein the inherent viscosity of each polymer is measured for a 0.1% w/v solution in chloroform at 25° C. The implant may sustain release of a therapeutically effective amount of the bimatoprost into an eye for a period of two months or longer. Table 1 below shows an exemplary formulation.

TABLE 1

| | | | | Quantity in |
|---|---|---|---|---|
| | Function | Formulation | Overage | a 40 gram |
| Material | Drug | (% w/w) | (%) | Batch (g) |
| Bimatoprost | Substance | 20^{a} | 5 | 8.51 |
| Resomer ® RG752S | Excipient | 40 | 0 | 16 |
| (PLGA) | | | | |
| Resomer ® R203S | Excipient | 20 | 0 | 8 |
| (PLA) | | | | |
| Resomer ® R202H | Excipient | 15 | 0 | 6 |
| (PLA) | | | | |
| PEG 3350 | Excipient | 5 | 0 | 2 |

In some embodiments, the intraocular implant is sized and formulated for placement in the posterior chamber of the eye. An implant sized for placement in the posterior chamber of an eye and capable of delivering a therapeutically effective amount of bimatoprost to the mammalian eye for an extended period according to this disclosure is generally from 20 μg to 200 μg in total weight, from 0.5 to about 3.0 mm in length, and from 0.1 to 0.5 mm in diameter (or other smallest dimension as may be appropriate for non-cylindrical implants). In some embodiments, an implant sized for placement in the posterior chamber (a posterior chamber implant) may weigh (therefore have a total weight) from about 30 to about 150 μg and contain from about 6 μg to about 30 μg of bimatoprost or other prostamide. In a preferred embodiment, the posterior chamber implant has a total weight of from 30 to 150 μg and is 150 μm to 300 μm in diameter and 0.5 mm to 2.5 mm in length. In a more preferred embodiment, the biodegradable posterior chamber implant according to this disclosure has a total weight of 30 μg to 100 μg and is 150 μm to 300 μm in diameter and 0.5 mm to 2.5 mm in length. In some embodiments, the implant is about 150 to about 300 μm in diameter or width, about 1.0 mm to about 2.5 mm in length, and about 30 μg to about 100 μg in total weight. In some embodiments, the implant is 150 to about 300 μm in diameter or width, 1.0 mm to 2.5 mm in length, and 30 μg to 75 μg, or 30 to 90 μg in total weight. The implant may be an extruded implant (i.e., the implant may be produced by an extrusion process). In some embodiments, the implant is formed by an extrusion process and is 150 to 300 μm in diameter or width, 0.50 to 2.5 mm in length, and 30 to 100 μg in total weight.

Thus, a posterior chamber implant according to this disclosure may have a total weight of from about 20-120 μg, 30-100 μg, 30-90 μg, 30-75 μg, or 30-50 μg. Non-limiting examples include extruded implants containing about 6 μg, 10 μg, 15 μg, or 20 μg (±5%) bimatoprost and having a total weight of about 30 μg, 50 μg, 75 μg, or 100 μg (±5%), respectively. In certain forms the extruded implant may have a diameter of about 200 μm or 250 μm (±5%) (before placement in the eye or other liquid or fluid environment) and a length of about 2.3 mm, 1.5 mm, or 1.0 mm (±5%). In one embodiment the posterior chamber implant is about 200 μm to about 300 μm in diameter, and about 1.0 to about 2.3 mm in length. An implant sized for placement in the posterior chamber of an eye according to this disclosure and according to any of the foregoing embodiments can comprise 20% (w/w) bimatoprost, 20% (w/w) 82035, 15% (w/w) R202H, 40% (w/w) RG752S, and 5% (w/w) polyethylene glycol (PEG) 3350.

Implants described here have the advantage of avoiding contact with the corneal endothelium due to their placement behind the iris thereby reducing the risk of corneal endothelial cell loss compared to implants positioned within the anterior chamber. Implants of the particular size, for example, no more than 3.0 mm long and no more than 0.5 mm maximum width, may have the additional advantage of fitting within the posterior chamber of the eye without significant contact with or chafing of the iris.

One embodiment is an extruded biodegradable intraocular implant according to this disclosure that is sized for placement in the posterior chamber of the eye, whereby the implant is 150 to 300 m in diameter, 0.50 to 3 mm in length, and 25 to 100 μg in total weight. Another embodiment is an extruded biodegradable intraocular implant according to this disclosure that is sized for placement in the posterior chamber of the eye, whereby the implant is 150 to 250 μm (±5%) in diameter, 0.75 to 2 mm in length, and 50 to 75 μg in total weight. The implant according to either embodiment will usually comprise 20% by weight bimatoprost as the active agent in association with a biodegradable polymer matrix comprising or consisting of i) an ester-end poly(D,L-lactide), ii) an acid-end poly(D,L-lactide), and iii) an ester-end poly(D,L-lactide-co-glycolide) having a D,L-lactide:glycolide ratio of about 75:25 and an inherent viscosity of 0.16-0.24 dl/g, wherein the inherent viscosity is measured for a 0.1% solution of the polymer in chloroform at 25° C. In a more specific embodiment, the ester end poly(D,L-lactide) has an inherent viscosity of 0.25-0.35 dl/g and the acid-end poly(D,L-lactide) has an inherent viscosity of 0.16-0.24 dl/g.

The size and geometry of the implant can also be used to control the rate of release, period of treatment, and drug concentration at the site of implantation. Larger implants will deliver a proportionately larger dose, but depending on the surface to mass ratio, may have a slower release rate. The particular size and shape of the implant are chosen to suit the site of implantation, and may also be consistent with the size of the needle used to place the implant into the eye.

The implants may be produced in a variety of shapes, including as a rod, sheet, film, wafer, or compressed tablet, but are preferably in the form of an extruded rod. An extruded rod may be cylindrical or non-cylindrical in shape. The implants may be monolithic, i.e. having the active agent or agents homogenously distributed through the polymeric matrix.

An implant according to this disclosure may desirably provide a substantially constant rate of prostamide release from the implant over the life of the implant. For example, it may be desirable for the prostamide to be released in an amount between 0.01 μg and 2 μg per day until 80-100% of the drug load has been released. However, the release rate may change to either increase or decrease depending on the formulation of the biodegradable polymer matrix. In addition, the release profile of the prostamide component may include one or more linear portions.

A therapeutically effective amount of bimatoprost for reducing intraocular pressure in an eye of a patient may correspond to a bimatoprost release rate in the eye of about 50 to 500 ng/day.

Release of the prostamide from a biodegradable polymer matrix may be a function of several processes, including diffusion out of the polymer, degradation of the polymer and/or erosion or degradation of the polymer. Some factors that influence the release kinetics of active agent from the implant can include the size and shape of the implant, the size of the active agent particles, the solubility of the active agent, the ratio of active agent to polymer(s), the method of manufacture, the surface area exposed, and the erosion rate of the polymer(s). For example, polymers may be degraded by hydrolysis (among other mechanisms), and therefore, any change in the composition of the implant that enhances water uptake by the implant will likely increase the rate of hydrolysis, thereby increasing the rate of polymer degradation and erosion, and thus, increasing the rate of active agent release. Equally important to controlling the biodegradation of the polymer and hence the extended release profile of the implant is the relative average molecular weight of the polymeric composition employed in the implant. Different molecular weights of the same or different polymers may be included in an implant to modulate the release profile.

The release kinetics of the implants described herein can be dependent in part on the surface area of the implants. A larger surface area may expose more polymer and active agent to ocular fluid, and may cause faster erosion of the polymer matrix and dissolution of the active agent particles in the fluid. Therefore, the size and shape of the implant may also be used to control the rate of release, period of treatment, and active agent concentration at the site of implantation. As discussed herein, the matrix of the intraocular implant may degrade at a rate effective to sustain release of an amount of bimatoprost or other prostamide for two months after implantation into an eye.

The release rate of an active agent, such as bimatoprost, from an implant may be empirically determined using a variety of methods. A USP approved method for dissolution or release test can be used to measure the rate of release (USP 23; NF 18 (1995) pp. 1790-1798). For example, using the infinite sink method, a weighed sample of the drug delivery system (e.g., implant) is added to a measured volume of a solution containing 0.9% NaCl in water (or other appropriate release medium such as phosphate buffered saline), where the solution volume will be such that the drug concentration after release is less than 20%, and preferably less than 5%, of saturation. The mixture is maintained at 37° C. and stirred slowly to ensure drug release. The amount of drug released in to the medium as a function of time may be quantified by various methods known in the art, such as spectrophotometrically, by HPLC, mass spectroscopy, etc.

The intraocular implants described here comprise a mixture of at least three different biodegradable polymers selected from the group consisting of poly(D,L-lactide) (PLA) polymers and poly(D,L-lactide-co-glycolide) (PLGA) polymers. Differences between the three polymers may be with regard to the end group, inherent viscosity, or repeating unit, or any combination thereof.

Poly (D,L-lactide), or PLA, may be identified by CAS Number 26680-10-4, and may be represented by the formula:

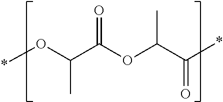

Poly(D,L-lactide-co-glycolide), or PLGA, may be identified by CAS Number 26780-50-7, and may be represented by the formula:

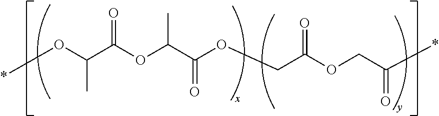

Thus, poly(D,L-lactide-co-glycolide) comprises one or more blocks of D,L-lactide repeat units (x) and one or more blocks of glycolide repeat units (y), where the size and number of the respective blocks may vary. The molar percent of each repeat unit in a poly(lactide-co-glycolide) (PLGA) copolymer may be independently 0-100%, 50-50%, about 15-85%, about 25-75%, or about 35-65%. In some embodiments, the D,L-lactide may be about 50% to about 85% of the PLGA polymer on a molar basis. The balance of the polymer may essentially be the glycolide repeat units. For example, the glycolide may be about 15% to about 50% of the PLGA polymer on a molar basis.

More specifically the at least three different biodegradable polymers included in an intraocular implant according to this disclosure are independently selected from the group consisting of:
a) a poly(D,L-lactide) having an acid end group and an inherent viscosity of 0.16-0.24 dl/g, as measured for a 0.1% solution in chloroform at 25° C. (such as for example R202H);
b) a poly(D,L-lactide) having an ester end group and an inherent viscosity of 0.25-0.35 dl/g, as measured for a 0.1% solution in chloroform at 25° C. (such as for example R203S);
c) a poly(D,L-lactide-co-glycolide) having an acid end group, an inherent viscosity of 0.16-0.24 dl/g (as measured for a 0.1% solution in chloroform at 25° C.), and a D,L-lactide:glycolide molar ratio of about 50:50 (such as for example RG502H);
d) a poly(D,L-lactide-co-glycolide) having an ester end group, an inherent viscosity of 0.16-0.24 dl/g (as measured for a 0.1% solution in chloroform at 25° C.), and a D,L-lactide:glycolide molar ratio of about 50:50 (such as for example RG502);
e) a poly(D,L-lactide-co-glycolide) having an ester end group, an inherent viscosity of 0.16-0.24 dl/g (as measured for a 0.1% solution in chloroform at 25° C.), and a D,L-lactide:glycolide molar ratio of about 75:25 (such as for example RG752S);
f) a poly(D,L-lactide-co-glycolide) having an ester end group, an inherent viscosity of 0.50-0.70 dl/g (as measured for a 0.1% solution in chloroform at 25° C.), and a D,L-lactide:glycolide molar ratio of about 75:25 (such as for example RG755S); and
g) a poly(D,L-lactide-co-glycolide) having an ester end group, an inherent viscosity of 1.3-1.7 dl/g (as measured for a 0.1% solution in chloroform at 25° C.), and a D,L-lactide:glycolide molar ratio of about 85:15 (such as for example RG858S).

Unless otherwise specified, the inherent viscosities of the PLA and PLGA polymers referred to in this disclosure are determined for a 0.1% (w/v) solution of the polymer in chloroform (CHCl3) at 25° C.

Biodegradable PLA and PLGA polymers, such as the RESOMER® Biodegradable Polymers R203S, R202H, RG752S, RG755S, and RG858S, are available commercially from sources such as Evonik Industries, AG, Germany (Evonik Rohm Pharma GmbH), and Sigma-Aldrich.

In addition to bimatoprost and the at least three different biodegradable polymers, some implants according to this disclosure further include a polyethylene glycol having a molecular weight of 300 Da to 20,000 Da. For example, an implant may comprise polyethylene glycol 3350 (PEG 3350), or alternatively polyethylene glycol 20,000 (PEG 20K).

The prostamide component of the implant may be in a particulate or powder form and it may be entrapped by, embedded within, or distributed uniformly or non-uniformly throughout the biodegradable polymer matrix. In the presently disclosed implants, the prostamide will usually comprise about 20% of the implant on a weight to weight (w/w) basis. In other words, the prostamide will constitute about 20% of the implant by weight. More generally, the prostamide can comprise (i.e., be present in an amount of or constitute) 18% and 22% of the implant by weight.

In addition to bimatoprost or other prostamide, the intraocular implants and other drug delivery systems (e.g., microspheres) disclosed herein may optionally include one or more buffering agents, preservatives, antioxidants, or other excipients, or combinations thereof. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents are advantageously present in amounts sufficient to maintain a pH of the system of between 2 to 9 and more preferably 4 to 8. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These buffering agents, preservatives, antioxidants, and other excipients may be present in amounts of from 0.001 to 10% by weight of the implant.

Examples of antioxidant agents include ascorbate, ascorbic acid, alpha-tocopherol, mannitol, reduced glutathione, various carotenoids, cysteine, uric acid, taurine, tyrosine, superoxide dismutase, lutein, zeaxanthin, cryptoxanthin, astaxanthin, lycopene, N-acetyl-cysteine, carnosine, gamma-glutamylcysteine, quercitin, lactoferrin, dihydrolipoic acid, citrate, vitamins E or esters of vitamin E, and retinyl palmitate.

An implant may comprise a prostamide compound (for example, bimatoprost), prostaglandin analogs (for example, travoprost, latanoprost), any nitric oxide donating prostaglandin analog or prostamide as the sole active agent or may comprise a combination of two or more prostamides or prostaglandin analogs.

The biodegradable implants may be sterilized by gamma or by electron-beam radiation and inserted or placed into the anterior chamber or vitreous body of an eye by a variety of methods and devices, including needle-equipped delivery devices capable of ejecting the implant into the ocular region of the eye. An effective dose of radiation for sterilization may be about 20-30 kGy.

Because of their ability to release a therapeutically effective amount of bimatoprost for an extended period (e.g., 60 days or longer including 3 months, 4 months, 5 months up to about 6 months), implants in accordance with this disclosure are expected to be capable of reducing intraocular pressure in a patient for periods at least as long as drug delivery occurs without the need for frequent intraocular injections or regular instillation of eye drops to the ocular surface as may be necessary with topical therapy. Accordingly, in some forms, the implants described here are used as monotherapy (i.e. used alone to control the IOP without the use of adjunctive antihypertensive eye drops) to reduce intraocular pressure in a patient and thereby treat an ocular condition as described herein. Nevertheless, an implant in accordance with this disclosure can, if desired, be used in dual therapy in conjunction with the same or different therapeutic agent that is applied topically.

The implant will preferably deliver a therapeutically effective dose of the prostamide to the eye(s) for at least two months after placement in the eye, and will reduce the ocular condition, or at least one sign or symptom, or risk factor associated with the ocular condition, for at least 1 month, or for at least 2, or 4 months, and preferably for at least 6, 12, 24 months or more, following placement of the implant in the posterior chamber of the eye. If desired, more than one implant can be placed in the eye. For example, two implants may be placed in the posterior chamber of the eye to deliver a larger dose of the prostamide. For example, in one method an eye may be dosed with 20 μg of bimatoprost, by placing two 10-μg implants (each containing 20% bimatoprost by weight) in the posterior chamber of the eye simultaneously rather than using a single 20-μg implant. In another example, in a different method an eye may be dosed with 12 μg of bimatoprost, by placing two 6-μg implants (each containing 20% bimatoprost by weight) in the posterior chamber of the eye simultaneously rather than using a single 12-μg implant. Using two smaller implants may possibly improve the tolerability of the implants in the eye. Some embodiments comprise placing two implants in an ocular region of the eye, such as, for example, the posterior chamber, anterior chamber, and/or vitreous body of the eye. Depending on the overall size of the implant within the lumen of the needle, the arrangement of delivery apparatus components can be adjusted to accommodate the presence of the multiple implants.

One embodiment is a method for reducing intraocular pressure in an eye of a mammal, the method comprising placing a biodegradable intraocular implant according to this disclosure in an eye of the mammal, whereby the implant provides a prostamide to the eye in an amount effective for reducing intraocular pressure in the eye. In some forms of this method the mammal is a human patient that has elevated intraocular pressure, ocular hypertension, or glaucoma, and the implant is placed in the posterior chamber of the affected eye(s) of the patient. According to some embodiments, the method is effective for the lowering of intraocular pressure (IOP) in patients with open angle glaucoma or ocular hypertension. In other embodiments, the methods are effective for the lowering of IOP in patients with open angle glaucoma. In some embodiments the methods are therapeutically effective for the lowering of IOP in patients with open angle glaucoma or ocular hypertension who are inadequately managed with topical IOP-lowering medication (e.g., due to intolerance or nonadherence) or are unsuitable for topical therapy. In some embodiments the methods are therapeutically effective for the lowering of IOP in patients with open angle glaucoma who are inadequately managed with topical IOP-lowering medication (e.g., due to intolerance or nonadherence) or are unsuitable for topical therapy. The implant may be effective for reducing intraocular pressure in the eye for at least two months after placement in the posterior chamber of the eye. In some instances, the implant may reduce intraocular pressure in the eye for greater than 12 months after placement of the implant in the eye. In some embodiments, a single implant may reduce intraocular pressure for between 12 and 24 months (see, e.g., US 2019/01923414 filed Nov. 8, 2018).

Anterior chamber delivery of implants can pose a risk for contact between the inner surface of the corneal covered by a delicate layer of endothelial cells. Contact with the corneal endothelium can reduce corneal endothelial cell density and onset of corneal edema in the eye. The implants described herein are sized for placement behind the iris within the posterior chamber of the eye, which mitigates the problem posed by contact between the implant and the corneal endothelium. Surprisingly, posterior chamber placement of the implant also enhanced IOP reduction achieved by the drug treatment compared to placement of the equivalent implant in the anterior chamber. The enhanced IOP reduction occurred even though drug release occurred further away from the aqueous outflow paths and having a reduction in drug concentration at the target tissue. Posterior chamber placement of bimatoprost implants therefore provides a safer and more effective IOP reduction compared to conventional anterior chamber placement of bimatoprost implants.

The present disclosure also provides for a method for reducing or lowering intraocular pressure in a patient, the method comprising placing a biodegradable intraocular implant in an eye of the patient, thereby reducing intraocular pressure in the eye for an extended period such as, for example, for at least one month, two months, or for at least four months. In some instances, the patient may have open-angle glaucoma, or more specifically primary open-angle glaucoma, and/or ocular hypertension. The implant used in the method can be any of the prostamide-containing implants described herein. In a preferred embodiment, the method comprises placing an extruded intraocular implant comprising bimatoprost in an eye of the patient. The implant can be placed in the posterior chamber of the eye, for example, in the ciliary sulcus of the eye, atop the zonules, etc.

An extended duration of IOP-lowering effect may also be observed with other dosing regimens of an intraocular implant as described herein to treat a patient with open-angle glaucoma or ocular hypertension. For example, a patient may receive 1 or 2 or 3 or 4 or 5 or 6 or 7 or 8 total implants over a treatment duration, with a single implant injected into the patient's posterior chamber once every 3 months (about 12 weeks) or 4 months (about 16 weeks) or 5 months (about 20 weeks) or 6 (about 24 weeks) or 7 months (about 28 weeks) or 8 months (about 32 weeks) or 9 months (about 36 weeks) or 10 months (about 40 weeks) or 11 months (about 44 weeks) or 12 months (about 48 weeks) and experience an increased duration of IOP-lowering effect and/or amount of time without the need for a rescue medication for reduction of IOP (e.g., prostaglandin analog or prostamide-containing eye drops such as latanoprost, travoprost, or bimatoprost). The duration of IOP-lowing effect or amount of time without the need for a rescue medication after such dosing regimen may be 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, or more than 24 months. In some embodiments, the duration of IOP-lowing effect or amount of time without the need for a rescue medication can be in the range of 12 months to 24 months, over 12 months to 15 months, 13 months to 24 months, over 12 months to 24 months, over 12 months to 16 months, over 12 months to 20 months, over 16 months to 24 months, and the like. In some embodiments, the duration of IOP-lowing effect or amount of time without the need for a rescue medication can be in the range of 12 months to 24 months, over 12 months to 15 months, 13 months to 24 months, over 12 months to 24 months, over 12 months to 16 months, over 12 months to 20 months, over 16 months to 24 months, after receipt of the final implant over the treatment duration.

Delivery Device

Described herein is an implant delivery apparatus that is designed specifically to access and deliver an implant into the posterior chamber, preferably within the ciliary sulcus. The delivery apparatus generally includes an appropriately sized shaft to minimize trauma to the eye (for example, a 25, 26, 27, 28, 29, or 30 gauge shafts). In some embodiments, the hand-held applicator comprises an 25 to 30 gauge stainless steel cannula, a lever, an actuator, and a push rod to promote ejection of the implant from the device into the eye.

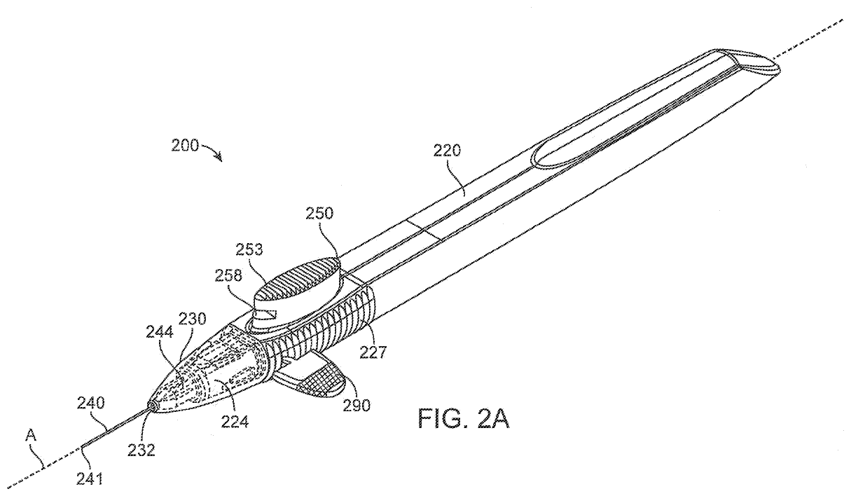

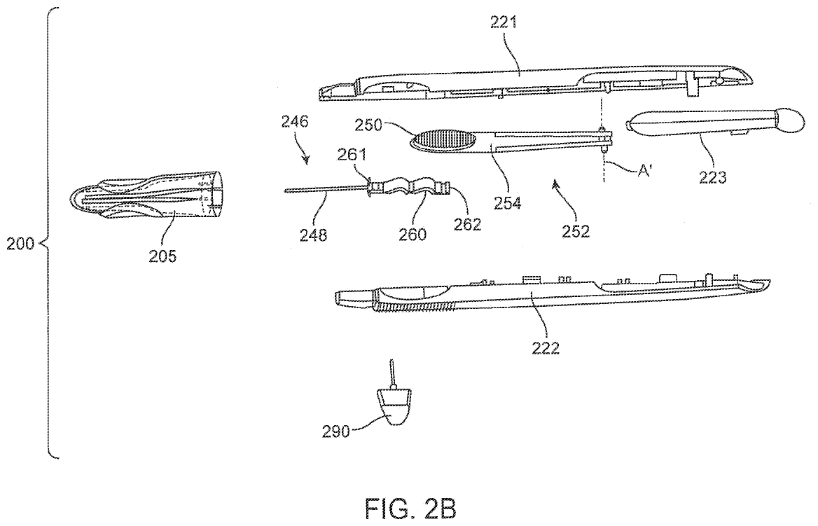

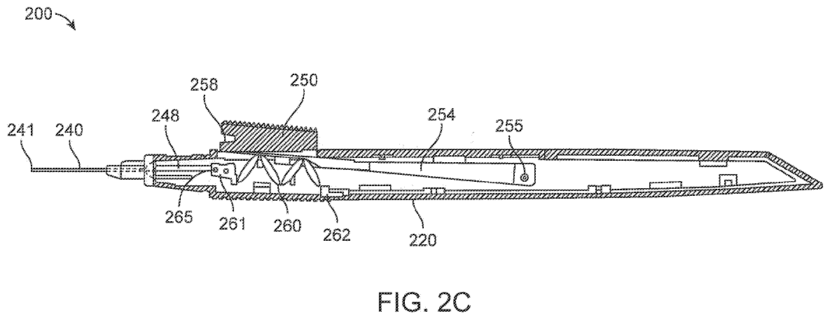

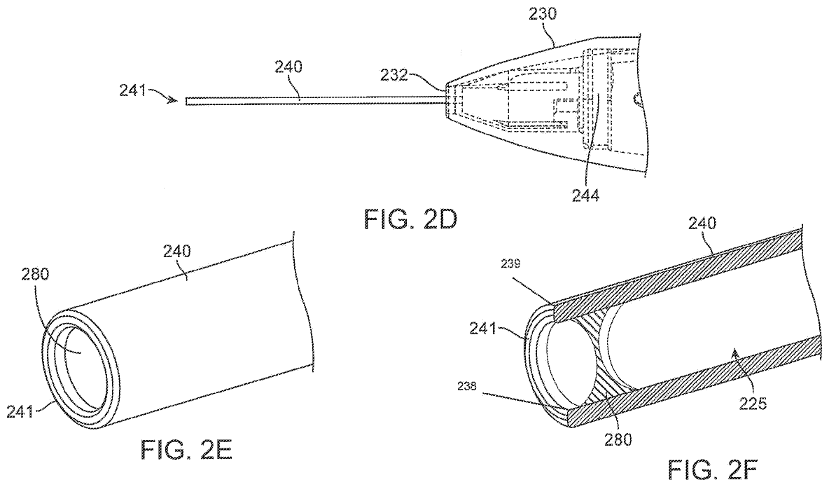

FIGS. 2A-2F illustrate an implementation of such an implant delivery apparatus 200. The delivery apparatus 200 can include a housing 220 with a nose cone 230 attached to and extending from a distal end region of the housing 220. The housing 220 is sized to be held in a single hand by an operator. The housing 220 can include an actuator such as an ejector button 250 configured to eject an implant loaded within the cannula 240. A cannula 240 can extend distally from the nose cone 230 and have an inner lumen sized to contain the implant. Preferably, the cannula 240 incorporates a retention plug 280 attached to the cannula so as to span the lumen inside the cannula 240, for example, near the distal opening, to prevent inadvertent premature release of the implant from the cannula 240 (see FIG. 2F). The intraocular implant can be positioned within the lumen of the cannula proximal to the retention plug 280. The cannula 240 has an outer dimension sized and an exposed working length specifically for ab interno deployment of the implant into the posterior chamber of a patient's eye, preferably through a self-sealing corneal incision or puncture. In some implementations, the cannula 240 is no larger than 28 gauge. The cannula 240 can extend along a longitudinal axis between a proximal end of the cannula 240 to a distal end of the cannula 240. The distal end forms a blunt tip 241 due to the inner and outer edges 238, 239 defining the blunt, non-beveled, distal opening from the lumen 225 of the cannula 240 being rounded. The blunt-tipped cannula 240 with rounded edges 238, 239 permits safe delivery of each implant 10 into the posterior chamber by reducing the risk of accidental trauma to the iris and lens capsule. The exposed working length of the cannula 240 beyond the nose cone 230 is longer compared to other delivery devices permitting for deeper insertion through the anterior chamber and pupil so that the distal end region of the cannula 240 is capable of reaching the posterior side of the iris. The cannula 240 is sized to allow about 1 mm to 2 mm of the distal end of the cannula 240 to be positioned behind the iris fringe for deployment of the implant.

The cannula 240 can be substantially straight along a longitudinal axis A of the apparatus 200 between the nose cone 230 to the distal tip 241 without bends or curves. The cannula 240 can be formed of a substantially rigid material and in a variety of small gauges, preferably up to 28 gauge having a nominal outer diameter (depending on wall thickness of the cannula) of about 0.362 mm and a nominal inner diameter of about 0.184 mm. The cannula 240 can be stainless steel or other medical grade material that does not interact with the drug product contained within the lumen of the cannula. The gauge is selected such that the inner diameter of the cannula lumen 225, or bore, will correspond to or accommodate the outer diameter of the chosen implant, with enough tolerance such that the implant can be received into and subsequently ejected from the cannula lumen 225. The cannula 240 can incorporate a standard surgical luer lock fitting on its hub, which can be received and secured to a corresponding luer fitting provided on the end of housing 220. Preferably, the cannula 240 is not a sharpened needle and instead has a blunt, atraumatic tip 241 that terminates at a distal opening from the lumen 225. FIGS. 2D-2F illustrate the tip 241 of the cannula 240 showing the rounded inner and outer edges 238, 239 that define the distal opening from the lumen 225 of the cannula 240 (see FIG. 2F). The rounded edges 238, 239 permit the cannula 240 to be inserted to the posterior chamber without jabbing into the anterior capsule or scraping the posterior surface of the iris. The iris is a fairly loose pigmented layer that is prone to sloughing with contact, particularly against square edges. The rounded edges 238, 239 also reduce snagging on the corneal stroma when entering through the anterior chamber.

The cannula can be substantially rigid and inflexible having a column strength that prevents the cannula 240 from flexing during advancement to the posterior chamber. The outside surface of the cannula 240 can be tumble polished, electropolished, or the equivalent for a smooth, atraumatic finish that does not catch or otherwise snag on eye tissue such as the corneal stroma or the lens capsule during advancement toward the posterior chamber. Conventional hypodermic needles can be coated with hydrophobic materials like silicone oil to improve glide through tissues. The outside surface of the cannula 240 can be coated such as with a silicone oil coating. This “siliconized” outside surface provides lubricity and a very low coefficient of friction for the surface of the cannula 240 that decreases risk of unintended trauma at the point of cannular insertion into the eye (e.g., the cornea). This, in combination with the rounded edges of the cannula tip, significantly improve the safety of implant delivery to the posterior chamber. The cannula 240 can be siliconized, preferably by spray-coating or electro spray coating, with silicone oil so that the external surface of the cannula 240 is preferentially siliconized and the inside surface (i.e., the lumen) is substantially non-siliconized, or siliconized only within a region near the opening into the lumen in trace amounts. Spray-coated siliconized cannulas can include a small amount of silicone migrating from the distal tip and entering the lumen, but not so much that the silicone coats a surface of the lumen where the retention plug may be positioned. Dip-coating with silicone can coat the outside surface of the cannula as well as allow silicone to enter the bore of the cannula via capillary forces and coat the inside surfaces of the cannula and thus is generally not a recommended method of applying silicone unless sufficient efforts are taken to prevent silicone from entering the lumen. This coating of the inside surfaces can be particularly problematic with larger needle sizes (e.g., 22 G). Siliconizing the lumen 225 of the cannula 240 can negatively impact the robustness of the retention plug 280 within the cannula 240 and increase the likelihood of plug failure or failure to retain the implant 10 within the lumen 225 prior to actuation. Spray-coating avoids substantially siliconizing the bore and focuses the silicone to the outside surfaces so that the silicone and the retention plug 280 do not come into substantial contact with one another. The retention plug 280 has improved adherence to the non-siliconized surface of the cannula bore 225 compared to the surface which has been coated with silicone. The implant retention plug 280 will be described in more detail below.

The silicone can cover the entire exposed working length of the cannula 240 near where a proximal end of the cannula 240 extends outside the nose cone 230 to the distal tip 241 of the cannula 240. For example, the silicone can cover the entire exposed outer surface of the cannula (e.g., about 12 to about 18 mm). The silicone can cover an exposed length of the cannula that is less than the entire exposed outer surface of the cannula 240. For example, the silicone can cover the distal end region along a length including the distal-most end of the cannula 240 to about 1.0 mm-about 5 mm away from the distal-most end. The silicone can coat the distal-facing surface of the cannula including the rounded outside edge 239 and the rounded inside edge 238 defining the opening (see FIG. 2D). Complete coverage of the surfaces of the cannula 240 that come into contact with eye tissue can improve insertion of the cannula through that tissue, for example, the blunt tip through the opening in the cornea, without snagging. In some implementations, the distal-facing surface of the cannula 240 and the rounded outside edge 239 are coated with silicone, but the rounded inside edge 238 is either not coated with silicone, or has a level of silicone that has been deemed to be insignificant and shown to not interfere with the ability of the retention plug to retain the implant within the lumen. Still further, the silicone coating can exclude both the inside and outside edges 238, 239 as well as the distal-facing surface of the cannula 240 such that the coating begins a short distance proximal to the distal-most end of the cannula 240. It should also be appreciated that the cannula 240 described herein can be free of any silicone coatings whether on the inside surfaces or the outside surfaces.

It is desirable, although not necessary, to use a cannula 240 that corresponds in dimensions to a 21 or 22 gauge shaft or smaller. The cannula 240 can have a gauge up to 28 G, preferably between 27 G and 28 G. Small cannula sizes (e.g., 27, 28, 30 g) have the important advantage that they can be received through a puncture or incision that according to techniques described herein are self-sealing and advanced into the posterior chamber of the eye without significant contact with or chafing of the iris. In the present application, this becomes advantageous in that the implant delivery into the eye can be accomplished without the need for suturing the puncture site, as would be necessary were a larger gauge used. Using a 21 or 22 gauge cannula or smaller, preferably 27 gauge or smaller, the implant can be placed and the cannula 240 withdrawn without excessive fluid leakage from the eye, despite the normal fluid pressures within the eye, and stitching of the puncture site can be avoided. Microimplants are dimensioned to have outer diameters to be received within the needle cannulaes with sufficient tolerances to be readily pushed through the cannula. For example and without being so limited, microimplants with a diameter of 0.018 inches can be easily delivered through a 22 gauge thin wall needle, and a microimplant with a diameter of 0.015 inches is easily deliverable through a 23 gauge thin wall needle. A microimplant with a diameter of 0.007 inches is easily deliverable through a 28 gauge cannula having a wall of 50 microns.

The wall thickness of the cannula 240 can be about 38 μm up to about 50 μm. In some implementations, the cannula 240 has a wall thickness that is generally not greater than about 50 m. The blunt-tipped cannula 240 is generally inserted through a pre-made opening in the eye and thus, need not have as much column strength as a tool designed to punch through a region of the eye without buckling. Thus, the cannula 240 can incorporate a thinner wall (i.e., about 38 μm) without risk of buckling. A 28 gauge cannula 240 having a wall thickness of about 50 m provides sufficient column strength for a posterior chamber approach without bending.

The exposed length of the cannula 240 between the nose cone 230 of the housing 220 and the distal tip 241 can vary. The cannula 240 can be inserted into the posterior chamber to a desired depth that is between about 12 mm to about 15 mm measured from the distal-most tip of the cannula 240 to the corneal surface where the cannula first penetrates the eye. The depth of penetration can vary and can be at least about 10 mm, at least about 11 mm, at least about 12 mm, at least about 13 mm, up to at least about 18 mm. In some implementations, the cannula 240 exposed working length between the proximal end and the distal end can be approximately 12 mm to 18 mm. In some implementations, the length of the cannula 240 is about 0.50″ (12.7 mm). In other implementations, the length of the cannula 240 is about 0.66″ (16.7 mm) or longer up to about 18 mm. The extended length of the cannula 240 compared to other devices for implant delivery in combination with the rounded distal edges and siliconization of the outside surface of the cannula 240 provide a safer and more reliable implantation of the implant to the posterior chamber.

The distal opening of the cannula 240 is distanced from the distal end of the nose cone 230 to allow for the implant to be delivered into the posterior chamber, preferably into a deep part of the ciliary sulcus near the iris root. The length of the cannula 240 is sufficient to extend from the external surface of the cornea near the limbus, across the anterior chamber, over the iris, and through the pupil into the posterior chamber, preferably into the ciliary sulcus. Generally, the distal opening from the cannula 240 is positioned near where the user desires the implant to be deployed. The distal opening from the cannula 240 can be positioned just past the iris fringe (e.g., about 1 mm to 2 mm past). The implant can be ejected a distance past the distal opening of the opening that is at least about 5 mm and no more than about 15 mm. As such the distal end of the cannula 240 in order to deploy the implant into the ciliary sulcus need only extend through the eye to a location that is no more than about 15 mm away from the target location of deployment, preferably posterior to at least a portion of the iris. The distal end of the cannula can slide just past the iris near a 6 o'clock position of the eye so that upon urging the implant out of the cannula the implant can float down over the zonules or into the ciliary sulcus. The distal end of the cannula need not be positioned within the ciliary sulcus in order for the implant to settle within the sulcus.

Again with respect to FIGS. 2A-2B, the apparatus 200 can be ergonomically configured for easy gripping and manipulation, and have a general overall shape similar to a conventional pen or other writing instrument. The apparatus will typically be grasped by the user between the thumb and the middle finger and can incorporate one or more features to improve grip and user comfort. For example, the housing 220 can include tactile ridges 227 in selected areas such as around the ejector button 250 where the thumb and middle finger of the user are in contact the apparatus, to provide a more secure grip and feel to the user. Ejector button 250 itself can be provided with tactile grooves 253 on the button surface where the finger (or thumb depending on preference) typically contacts the button, also providing for a more secure grip and feel for the user. The ridges 227 can be rubberized for comfort and to provide non-slip surfaces by which to firmly grip and hold the device. The ejector button 250 preferably incorporates no stored energy so that the user can fully control the speed and distance the implant extends out of the distal opening of the cannula. The slower the ejector button 250 is pressed, the slower the implant advances, and the more controlled the ejection of the implant from the cannula.

The housing 220 can be formed of two half sections 221 and 222. These sections are preferably configured to snap-fit together, although other known methods of attaching the two halves together are contemplated, including, e.g., gluing, welding, fusing, etc. Alternatively, the housing could be singularly molded. Label plate 223 can also be provided, which likewise can be snapped onto or otherwise secured to, the housing.

The distal nose cone 230 can be integral with the housing 220 or can be manufactured as a separate piece that is secured to the housing 220. Specifically, nose cone 230 can be secured to collar 224 of housing 220. Nose cone 230 can receive cannula assembly including cannula 240 and cannula hub 244. The hub 244 is configured for receipt and securement within nose cone 230 with cannula 240 extending through nose cone hole 232. The cannula lumen 225 is in communication with an inner passageway of the hub, such that implant can be passed through the inner passageway of the hub 244 and loaded into the cannula lumen 225. Because the implant 10 is pre-positioned within the lumen 225 no transfer step is necessary in order to deploy the implant 10 using the apparatus 200. The cannula lumen 225 can be sized to receive at least a portion of a plunger 246. The plunger 246 can include a push rod 248 extending distal to a linkage 260. The push rod 248 is configured for slidable receipt within the cannula lumen 225, and is of sufficient length to displace a loaded implant retained with the cannula lumen 225 and eject it from the cannula tip 241. The cannula 240 can include a tubular wall extending along a longitudinal axis between a proximal end of the tubular wall that is coupled to the housing such that it extends outside the nose cone and a distal end of the tubular wall. The tubular wall of the cannula defines the lumen, which is sized to slideably receive the push rod 248. The actuator on the housing can move the push rod through the lumen of the cannula to push the implant out from the lumen via the linkage. The actuator is coupled to the push rod through the linkage, the push rod being movable along the longitudinal axis as the linkage is gradually flattened as the actuator is depressed. The push rod can have a length relative to a length of the cannula sufficient for a distal end of the push rod to advance past the distal end of the cannula upon deployment of the implant using the actuator. In some implementations, the push rod 248 can project beyond the distal tip 241 to aid in deploying the implant from the blunt distal tip 241. The distal end of the push rod 248 can project beyond the distal tip 241 by about 0.5 mm to about 1.0 mm. For example, the push rod 248 can be 27.64 mm so that upon actuation of the device and full extension of the push rod 248 through the shaft at least 0.5 mm of the push rod 248 extends distal to the distal-most tip of the cannula 240. The length of the push rod 248 can vary and need not project beyond the distal tip 241 to deploy the implant. In some implementations, the push rod 248 is shortened to slow an ejection speed of the implant, which will be described in more detail below.

FIGS. 2B-2C illustrate actuating lever 252 and linkage 260, which can be retained within housing 220. Actuating lever 252 can include elongate member 254 having one or more pins 255 extending from the member 254 at one end and ejector button 250 extending from the other end. Pins 255 can extend along an axis A′ that is normal to the longitudinal axis A of the cannula 240. The pins 255 can be received in corresponding pivot holes (not visible) of the housing sections 221, 222, such that when assembled, the lever 252 can pivot about the pins 255 in a restricted range of motion within the housing 220.

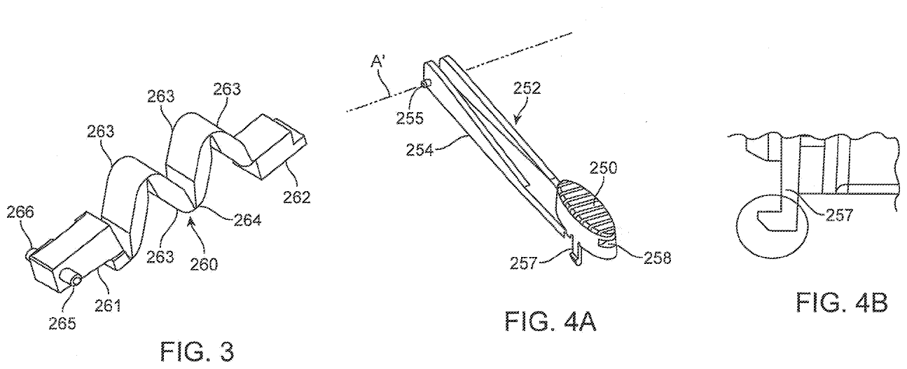

FIG. 2C and also FIG. 3 shows the linkage 260 can include front and rear blocks 261 and 262, with a plurality of joined segments 263 extending therebetween. The segments 263 are sequentially joined to one another. Flexible joints 264 connect the segments 263 to each other and to the front and rear blocks 261, 262. The linkage 260 is flexible yet resilient, and preferably formed of a contiguous, moldable plastic piece. Portions of the linkage 260 having a relatively thin cross-sectional area of material form flexible joints 264, and disposed between thicker, less flexible segments 263. This allows for flexure of the linkage 260 at the joint locations when a force is applied to the linkage 260. Other known materials are also suitable for the linkage 260, including e.g. shape memory alloys, provided the resultant linkage is capable of lengthwise extension when a force normal to or perpendicular to the length of the linkage is applied. When assembled, rear block 262 is fixedly secured into the housing 220. Guide pins 265, 266 can extend from front block 261 and be received within a guide track of the housing 220. The linkage 260 interacts with the button 250 in a manner that allows a user to depress the button 250 slowly so that advancement of the push rod along the longitudinal axis of the cannula 240 is gradual with depression of the button 250 that slowly flattens the linkage 260 to avoid shooting the implant out of the lumen 225 of the cannula 240 at a high velocity.

The underside of button 250 of actuating lever 252 can be in contact with the linkage 260 (see FIG. 2C). In operation, depression of button 250 by the user transmits force against the linkage 260 through underside of button 250 in a direction generally normal to the longitudinal axis A of the apparatus. This force is transmitted through the linkage 260, and is converted into a longitudinal force along the longitudinal axis A of the apparatus 200, through flexure of the linkage joints. Because the rear block end 262 of the linkage 260 remains fixed to the housing 220, this action results in translational motion of the free, front block end 261 of the linkage 260 in the direction away from the fixed rear block 262 of the linkage 260. This translational movement of the front block 261 of the linkage 260, in turn, pushes push rod 248 through the lumen 225 of cannula 240. Where an implant is loaded and retained within the cannula lumen 225, the motion of the push rod 248 in turn ejects the implant from the cannula tip 241.

FIGS. 4A-4B show the button 250 can also include a tab 257 extending below a plane of a lower surface of the button 250. The tab 257 can engage with tab slot (not shown) of the housing 220. The engagement between the tab 257 and the tab slot can retain the actuating lever 252 in a locked, depressed condition, after deployment of the implant 10. The tab 257 can include a detent which, when engaged in slot will provide an audible click, signaling the user that the implant has been deployed from the apparatus 200. In other implementations, the actuation of the button 250 provides some tactile feedback to the user that is inaudible or only somewhat audible to the patient so as to avoid causing inadvertent movements by the patient during deployment of the implant. The geometry of the lower surface of the tab 257 can be rounded and the tab 257 can be moveable so that it flexes inward as the tab 257 moves downward through the housing back the tab slot. Once the tab 257 moves a distance past the tab slot, the tab can return to its natural position and engage with the tab slot. The geometry and motion of the tab 257 relative to the tab slot provides an engagement that is dampened so as to provide at least some audible and tactile feedback to a user that the push rod 248 is in a fully deployed configuration relative to the cannula 240 without a startling snap.

The actuator 250 of the delivery apparatus 200 can vary including a plunger, slider, pushbutton, lever, or other actuator configured to cause the implant to be deployed within the eye. The actuator 250 can be a push button as shown in FIG. 2C that can activate a lever that drives a plunger or push rod 248 or other suitable means within the cannula 240 forward. As the push rod 248 moves forward, it can push the implant out of the lumen 225 of the cannula 240 into the target area. In other implementations, the delivery apparatus 200 can include a stopper that can remain in place and the cannula 240 is withdrawn relative to the stopper to deploy the implant out of the lumen 225. In still further implementations, the delivery apparatus 200 can incorporate a push-pull configuration in which both the outer cannula 240 and inner stopper are movable relative to one another.

The method of implantation and the devices used to position the implant can vary depending on whether the patient is phakic or pseudophakic. For example, pseudophakes may be implanted with an applicator having a needle tip that is sharp, whereas it is preferable, although not required, to use a blunt cannula tip for phakic patients to avoid inadvertent damage to the natural lens.

Implants that are compatible with loading and ejection from apparatus described herein can be formed by a number of known methods, including phase separation methods, interfacial methods, extrusion methods, compression methods, molding methods, injection molding methods, heat press methods and the like. Particular methods used can be chosen, and technique parameters varied, based on desired implant size and drug release characteristics. For microimplants described herein, which can be delivered through cannulas corresponding to a 22 gauge shaft or smaller extrusion methods are particularly useful. Extrusion methods, as well as injection molding, compression molding, and tableting methods, can all achieve the small cross-sectional diameters or areas required of microimplants. Extrusion methods also may result in more homogenous dispersion of drug within polymer, which can be important given the small dimensions of microimplants.

The apparatus 200 can be packaged to include a safety cap 205 extending over the cannula 240 and secured to the housing 220 (see FIG. 2B). This will provide a measure of safety during handling of the apparatus 200. The button 250 or other depression mechanism of the apparatus 200 can include a notch or slot 258 that receives the rim of the safety cap 205. In this configuration, the safety cap 205 will then also operate to guard against unintentional depression of the button 250 or other depression mechanism and ejection of the implant 10.

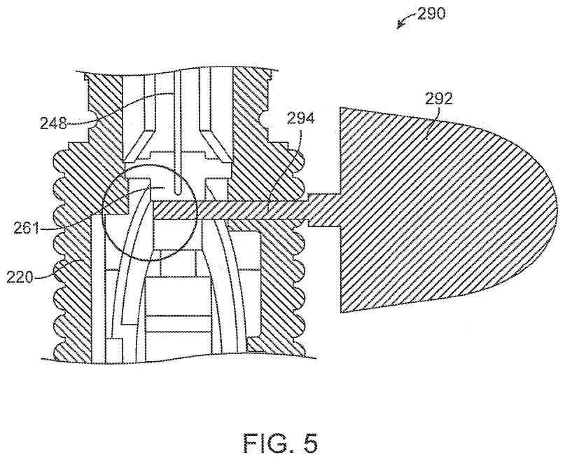

The apparatus 200 can incorporate a safety tab 290 configured to prevent inadvertent actuation of the button 250 (see FIG. 5). The safety tab 290 can include an external gripping portion 292 and an internal post 294. The internal post 294 is configured to prevent actuation of the linkage 260 by the button 250. In an implementation, the post 294 can engage with a region of the front block 261. When the post 294 is engaged with the front block 261, the translational movement of the front block 261 is prevented. Prior to injection, a user can grip the external portion 292 pulling it outward away from the housing 220 disengaging the post 294 from the block 261. The block 261 can then translate upon actuation of button 250. The safety tab 290 can have a discernable color or marking that alerts a user to its presence and that it should be removed prior to actuation of the button 250. For example, the tab 290 can have a first color and the housing 220 can have a second, different color. The tab 290 can also be labeled with words or symbols such as an arrow guiding a user in the direction the tab 290 should be pulled away from the housing 220.

When the apparatus is assembled with the implant, the implant be positioned immediately proximal of the opening at the cannula tip. In this fashion, the introduction of air into the eye can be avoided when the implant is ejected, as could otherwise occur were the implant located further within the cannula lumen and an air bubble or air pocket allowed to exist between the cannula tip and the implant and ejection of the implant were to force the air bubble or air pocket into the eye. One method to accomplish this is to load the implant distally into the cannula 240 followed by the plunger 246, with the push rod 248 length designed to push the implant to the desired pre-actuation position. When the cannula assembly is then installed onto the housing 220, the push rod 248 and thus the implant is advanced to the desired position. The push rod 248 can have a length sufficient to extend through the cannula 240 and make contact with the implant 10 to urge the implant 10 from the cannula 240. In some implementations, the push rod 248 can have a length sufficient to extend at least partially beyond the distal tip of the cannula 240 during actuation so that the implant 10 is expelled entirely from the bore and away from the cannula 240. The push rod 248 can extend out the opening so that the distal-most end of the push rod 248 projects beyond the distal-most end of the cannula 240. This allows the push rod 248 to drive the implant 10 well away from the tip 241 of the cannula 240 and into the fluid-filled medium of the eye (e.g., the posterior chamber, anterior chamber, or vitreous), the implant 10 is less likely to adhere to the end of the cannula 240 and thereby the chance that an implant is dragged out of the eye or becomes lodged in the tissues (e.g., cornea or choroid) when the shaft is removed. Deposition of the implant in the corneal endothelium, for example, may result in adverse complications. Implants preferably separate from the tip 241 immediately after ejection. The present device can provide for clean separation of the implant from the device since the push rod 248 may not only drive the implant through the lumen of the cannula but may also drive the implant away from the cannula tip.

The retention plug 280 can effectively retain the implant within the cannula 240, without significantly increasing the ejection force to deploy the implant and also without changing an outer dimension of the cannula 240. Minimizing the outer diameter of the cannula 240 means the opening into the eye is also minimized. The retention plug 280 can be robust enough to retain the implant within the cannula 240 during routine handling of the device, and still allow the implant to be ejected from the device without damaging the implant. The retention plug 280 can be formed so that it is positioned immediately proximal of the opening at the cannula tip 241 and the implant positioned immediately proximal to the plug 280. As discussed above, this prevents the presence of an air pocket existing between the distal end of the plug 280 and the tip of the cannula. The retention plug 280 retains the implant prior to ejection with reduced risk of premature implant ejection.

The length of the push rod 248 in cannulas incorporating a retention plug 280 can be shortened to accommodate the presence of the plug. The retention plug 280 can be positioned just proximal to the opening from the cannula 240 so the plug 280 is fully inside the bore behind the opening and the implant 10 can be positioned proximal to the plug 280. The distance the push rod 248 extends through the bore may be shortened by the length of the plug 280, for example, between about 1 mm-2 mm. In some implementations, the implant 10 can be about 7 mm in length and the push rod 248 can be long enough so that the push rod 248 contacts a proximal end of the implant 10 to urge the implant 10 out the opening from the bore. In other implementations, the plug 280 can be about 1 mm in length, and the push rod 248 can be about 27 mm allowing additional space for the plug 280. As used herein, a “shortened” push rod is a push rod that has a length less than a standard length push rod in order to accommodate the presence of the plug within the bore while also maintaining the relative positions of the implant and the push rod as in the applicator with a standard length push rod. Thus, the shortened push rod is shorter in length by an axial length of the plug spanning the lumen of the cannula. The degree by which the push rod is shortened is related to a dispensed mass of the retainer solution into the bore and the axial length of the resulting plug within the bore. The shortened push rod in an applicator incorporating a retention plug 280 can be shorter than a push rod of a standard applicator without a retention plug 280 by about 1 mm up to less than 2 mm.

The relative length of the push rod 248 to the cannula 240 can be varied in order to control the force and speed of the implant ejection thereby minimizing potential tissue damage. In some implementations, the push rod 248 has a length configured to extend out the distal end of the cannula 240 upon deployment of the implant so that the distal end of the push rod 248 is positioned past the rounded inner and outside edges of the cannula 240. In other implementations, the push rod 248 has a length configured to extend near the distal end of the cannula 240 upon deployment of the implant, but does not extend past the distal edges of the cannula 240. The push rod 248 in some implementations upon full actuation extends past the distal-most tip of the cannula 240. In other implementations, the push rod 248 upon full actuation stops short of the distal-most tip of the cannula 240 and does not extend past the distal-most tip of the cannula 240. For example, the push rod 248 can be a shortened push rod 248 (by less than about 2 mm down to about 1 mm shorter than a standard push rod) to accommodate the presence of a retention plug 280 within the bore. In this implementation, the push rod 248 may stop short of the distal-most tip upon complete actuation of the push rod 248. Prior to deployment, the push rod 248 can be separated a distance away from the implant 10 positioned in the bore. The distal end of the push rod 248 can be separated from the proximal end of the implant by about 1 mm up to about 2 mm. The shorter push rod 248, the increased distance between the push rod 248 and the implant 10, and the presence of the plug 280 can improve implant ejection performance compared to applicators incorporating longer push rod 248 and no plug 280. The length of the push rod 248 and/or the presence of the plug 280 can slow the ejection speed of the implant while maintaining a sufficient ejection distance in the eye to provide a reliable and safe deployment of the implant within the eye. The push rod can be shortened to accommodate the plug 280 such that the ejection speed of the implant is slowed to avoid striking delicate tissues upon actuation from the device.

The retention plug 280 can be formed of a single polymer or a mixture of two or more polymers. The retention plug 280 can be formed from a retainer solution containing a water-soluble polymer. The water-soluble polymer can be a cellulose ether, such as hydroxypropyl methyl cellulose “HPMC”. HPMC is commercially available, for example, from DuPont Pharma under the brand name METHOCEL®, and WALOCEL™, as well as from Ashland under the brand name BENECEL™. HPMC is also available in different grades having different viscosities based on the nominal methoxy and hydroxypropoxyl substitutions. The HPMC can be F4M grade HPMC having viscosity grade of 4,000 mPa and 27.0%-30.0% methyoxyl substitution (e.g., 29.0%) and 4.0%-7.5% hydroxypropoxyl substitution (e.g. 6.0%). The F4M grade HPMC can have a viscosity at 2% in water at about 20° C. that is between 2,663-4,970 cP. The HPMC can be E4M grade having viscosity grade of 4,000 cP and 28.0%-30.0% methoxl substitution. The E4M grade HPMC can have an apparent viscosity at 2% in water at about 20° C. that is between 2,663-4,970 cP. Other suitable grades of HPMC include A4M, E4M, F4M, K4M, and J4M, including any of the other grades within the A, E, F, K, and J series. The polymer of the retention plug may also be formed from any of a variety of water-soluble polymers including HPMC as described above, hydroxypropyl cellulose (HPC), polyvidone or povidone, hypromellose acetate succinate (HPMCAS), copovidone, crospovidone, methyl cellulose (MC), methylhydroxyethylcellulose (MHEC), sodium carboxymethyl cellulose (CMC), ethylcellulose (EC), hydroxyethylcellulose (HEC), hydroxypropyl-γ-cyclodextrin, hydroxypropyl-β-cyclodextrin, native cyclodextrin, N-methyl-2-pyrrolidone (NMP), hyaluronic acid, polyethylene oxide, polypropylene oxide, chitosan, agarose, polypeptide, ficoll, hydroxy ethyl methyl cellulose (HEMC), Poly (ethylene glycol) (PEG), N-(2-Hydroxypropyl) methacrylamide (HPMA), Polyoxazoline, Polyphosphates, Polyphosphazenes, Xanthan Gum, Pectins, Dextran, Albuminor natural and synthetic protein. The retainer solution containing the polymer for forming the retention plug 280 can form viscous solutions having an apparent viscosity preferably between about 6,000 cP and about 13,000 cP, having reasonable adhesion to non-siliconized metals, be water soluble, and biocompatible for use in the eye without causing adverse events. The specification sheets, data sheets, and testing data of these polymers are herein incorporated by reference in their entirety.

The retention plug can be formulated into a polymer retainer solution or polymer retainer gel before it is applied to a cannula. The polymer retainer solution or polymer retainer gel can contain the polymer as well as additional excipients for suitable purposes. In an embodiment, certain excipients may be added to the polymer retainer gel or the polymer retainer solution to modify the viscosity of the solution or gel, so that they can be easily applied to the implant administration device. According to an embodiment, an excipient comprising, consisting essentially of, or consisting of isopropyl alcohol and/or water and/or a buffer can be added to the polymer to form the polymer retainer solution or the polymer retainer gel. When used, one or more excipient can be present in the solution or gel in an amount in the range of 0.2% to 10% by weight of the solution or gel, based on the total weight of the solution or gel, and preferably about 3%.

Whether or not a plug forms in a needle bore can be a function of the concentration of the HPMC solution. In some implementations, the polymer retainer solution is an HPMC solution in water for injection (WFI) that creates different concentrations of liquid HPMC solutions between 2% w/w and 4% w/w, or between 2.5% w/w and 3.5% w/w, or preferably around 3% w/w. Concentrations as low as 1% solution fail to fully close off the lumen in the cannula whereas the viscosity of high concentration solutions (e.g., 6%) prevents dispensing within preferred dispense time, dispense pressure, and dispensing needle gauge parameters. The polymer retainer solution is preferably a solution of HPMC (e.g., E4M or F4M) that has a concentration greater than 2.5% and less than about 4%.

The different concentrations of HPMC solution can have different apparent viscosities. Apparent viscosity of the solution can be determined by rotational viscometry using a Brookfield RVDV-II+ Pro Extra viscometer and a Brookfield spindle #7-RV with a SC4-13RP sample chamber. The measurements can be taken at 100 rpm at a controlled temperature of 25.0±0.1° C. The tightly capped samples and standards (15 mL) can be placed in a water bath or oven/incubator and stabilized at temperature for at least 3 hours for equilibration prior to testing. In an implementation, a 2.58% F4M-HPMC solution has an apparent viscosity of 6,780 centipoise (cP), a 2.8% F4M-HPMC solution has an apparent viscosity of 7,880 cP, a 3% F4M-HPMC solution has an apparent viscosity of 10,680 cP, 3.15% F4M-HPMC solution has an apparent viscosity of 12,780 cP. The HPMC solution can have an apparent viscosity greater than or equal to about 6,240 cP and less than or equal to about 12,870 cP, greater than or equal to about 6,780 cP and less than or equal to about 12,760 cP, greater than or equal to about 8,640 cP and less than or equal to 10,080 cP prior to application to the cannula. In some implementations, the viscosity of the retainer solution is between about 7,000 cP and about 13,000 cP. In some implementations, the retainer solution is 3% F4M-HPMC solution having an apparent viscosity of 8,640 cP-12,760 cP dispensed into a 28 gauge cannula as a dispensed mass that is between 125 μg-200 μg.

The HPMC solution can be applied to a cannula having different gauges including 22 gauge, 25 gauge, 27 gauge, 28 gauge, or 30 gauge. Generally, the larger the bore, the more HPMC mass is dispensed to form the plug that is contained within and spans the bore. Cannulas that are 28 G may receive a dispensed mass of 100-300 μg, preferably about 125-275 μg, whereas cannulas that are larger (e.g., 22 G) may receive a larger dispensed mass of 300-1000 μg, preferably about 450-850 μg. It should be appreciated that other water-soluble, biocompatible polymers are considered herein for the polymer retainer solution for forming the retention plug. The concentration of polymer retainer solution can be selected to have an apparent viscosity in the ranges described herein and dispensed within the bore at a dispensed mass range described herein as being suitable for the particular cannula gauge.

Actuation force can be measured to determine how much force is required to press the actuator button to achieve deployment of an implant from the cannula. The actuation force of an applicator can be assessed by arranging the applicator in the horizontal position with the button pointing upwards by a fixture under force measuring equipment probe. The fixture keeps the applicator from deforming during the test so that actuation force on the button is measured accurately. The equipment probe can be connected to a force gauge so it pushes down onto the button of the applicator. The force in pounds (lbf) required to cause the button to travel downward into the housing of the applicator can be recorded by the equipment. The viscosity ranges described above for the retainer solutions to form a plug within the cannula resulted in actuation force to eject the implant that are comparable between 0.9 lbf and 1.5 lbf Generally, an actuation force that is below 5.0 pounds force is preferred.

The polymer retainer can be applied to the cannula as a solution, gel, or hot melt either by dipping the cannula tip into solution or by direct application of the polymer retainer to the tip of cannula. In embodiments where the polymer retainer is applied to the cannula tip as a solution or gel, the solution or gel solvent can be removed before the cannula containing the polymer retainer is inserted into a patient. In embodiments where the polymer retainer is applied as a hot melt, the melt can be cooled after application to the cannula, but before the cannula containing the polymer retainer is inserted into a patient.

Preferably, the cannula is not coated with the polymer retainer solution by dipping, but rather an amount of the solution is dispensed within the cannula bore by a dispensing tip of a controlled dispensing system to achieve a dispensed mass. The controlled dispensing system can be an EFD Ultra 2400 Fluid Dispenser, for example. The dispense tip ID range can be between about 0.00350″ and 0.0050″. Generally, the dispensing tip has a gauge that is smaller than the gauge of the cannula being plugged. For example, the cannula being plugged can be 28 gauge and the dispensing tip can be 32 gauge. The cannula being plugged can be 22 gauge and the dispensing tip can be 27 gauge.

A dispensed mass of the retainer solution can be deposited directly into the distal end of the cannula using the dispense needle and according to parameters such as polymer concentration, retainer solution viscosity, dispense needle gauge, applicator gauge, dispense time, dispense angle, dispense pressure, etc. to achieve a desired dispensed mass suitable for achieving a retention plug the fully plugs the bore of the cannula. The dispensed mass of the retainer solution can vary depending upon the concentration and apparent viscosity of the retainer solution as well as the cannula gauge being plugged. In some implementations, the dispensed mass of the retainer solution having an apparent viscosity of between 8,640 cP-12,760 cP to plug a 28 gauge cannula can be greater than 100 μg and less than 300 g, and preferably is in a range of about 125-275 μg. The dispensed mass of the retainer solution having an apparent viscosity of about 10,080 cP to plug a 22 gauge cannula can be greater than 300 μg and less than 1,000 μg, and preferably is in a range of about 450 μg and 850 μg.

The dispense parameters can vary depending on the cannula gauge being plugged and the solution being dispensed. The dispense pressure used to deliver the mass can be between 30 psi and 75 psi, preferably between about 50 and 55 psi. The dispense time can be between 1.0 and 3.0 seconds, preferably between about 1.4 and 2.8 seconds. The dispense angle can be between about 5 degrees and about 45 degrees. For a blunt-tipped cannula the dispense angle can be relatively shallow (e.g., less than about 10 degrees horizontal) whereas a needle having a bevel can have a greater dispense angle (e.g., about 30 degrees). In an implementation, for a viscosity range of retainer solution that is between 6,780 cP-7,880 cP and a dispensing tip ID range that is 90-100 μm, the pressure range can be between 47-51 psi to achieve a dispensed mass of between 125-275 μg within a 28 gauge cannula.

The solution following dispensing can be left to dry (e.g., at room temperature) to form a plug within the bore. Generally, the plug is formed proximal to the distal opening and fully inside the bore. The plugging parameters are selected to ensure complete plugs are formed that fully span the bore of the cannula as opposed to simply coating the walls. Preferably, the cannula is siliconized with electro sprayed-on silicone oil so that the silicone coats the outside surface and avoids siliconizing the inside surface of the cannula where the retention plug is to adhere or attach, as discussed elsewhere herein. The retainer plug attaches better to a non-siliconized surface. However, the retention plug can attach too well and negatively impact the functionality of the applicator (e.g., increasing actuation force to deploy the implant). Thus, the siliconization process allows for small amounts of silicone oil to migrate within the opening by capillary force. The spray-coating fully siliconizes the outside surface of the cannula and just a small amount of silicone oil settles on the opening of the lumen. The amount of silicone oil that migrates from the opening into the lumen in cannulas that are spray-coated is less than the amount of silicone oil that migrates in dip-coated cannulas thereby providing better adherence of the polymer retainer plug to the wall than dip-coated cannulas, but does not adhere so strongly that the plug impacts the usability of the device like the fully non-siliconized needles. The silicone coating can cover the entire exposed length of the cannula (about 6 mm along an outer surface) or length that is less than the exposed length of the cannula as described elsewhere herein.

A retention plug that retains an implant inside the cannula can vary in its effect on actuation force. Relevant parameters that can impact the suitability of a retention plug as a retention system for an applicator and its impact on actuation force from the cannula can vary including molecular weight of the polymer, concentration and viscosity of the retainer solution as well as the dispensed mass of the deposited retainer solution, which can vary depending on the inner dimension, dispense pressure, and dispense time of the controlled dispensing system used. The dispensed mass can be impacted by the retainer solution concentration and/or viscosity. For example, a higher concentration retainer solution may have a viscosity that the dispense pressure, dispense time, and/or inner dimension of the dispensing needle may need to be modified to achieve the same dispensed mass of a lower concentration retainer solution within the same size cannula. Some retainer solutions are too viscous for a particular cannula size and can form a plug that is too hard and increases the actuation force needed to deploy the implant that the device is not suitably usable. Other retainer solutions are not viscous enough for the cannula size used and form a plug that only coats the inner walls of the cannula leaving the aperture at least partially open and/or that fails to prevent the implant from falling out during transportation and handling. The viscosity of the retainer solution can be adjusted to provide different functional characteristics to the resulting plug that may be selected depending on the size of the cannula to be plugged. The viscosity of the retainer solution can be lower for a smaller gauge cannula and still sufficient to plug the opening to suitably retain the implant without impacting actuation force, but the same viscosity retainer solution may not be sufficient to plug the opening of a larger gauge cannula and instead merely coat the inner walls of the cannula or fail to retain the implant. And still further, an HPMC solution may have a viscosity sufficient to fully close the opening of the cannula, but be so viscous that an overly rigid plug forms such that the actuation force needed to push the implant through the plug is too high.

Viscosity of the retainer solution can be adjusted by changing the concentration of the retainer solution and/or the molecular weight of the polymer. For example, an HPMC starting material having a higher molecular weight may be selected to create an HPMC solution that is more viscous and suitable for a larger size cannula, but that may be too viscous for a smaller size cannula. The viscosity of the retainer solution can be lowered by selecting a lower molecular weight HPMC such that the same concentration retainer solution is suitable for the smaller size cannula, but that may not be suitable for the larger cannula size. Viscosity that is too low prevents the plug from effectively retaining the implant within the cannula, for example, during transportation and handling. The plug may also fail to sufficiently slow the implant as it is ejected from the cannula and enters the eye. An implant that shoots out of the cannula too quickly can travel too far into the eye causing tissue damage. Thus, the plug is preferably formed from a retainer solution that has a viscosity suitable for a particular cannula size in order to retain the implant during handling and also slows down the implant under the same ejection force as it is ejected from the cannula to avoid damaging the implant and the eye. A suitable amount of polymer retainer may be used to effectively plug the cannula to contain the implant, but still beneficially minimize the amount of actuation force necessary to expel the implant from the cannula. The polymer retainer plugs described herein can provide suitable retention and adherence with the bore, but do not significantly increase the actuation force required to deploy the implant from the cannula. The actuation force on the button to deploy the implant from the cannula can be in the range of about 0.5 pound force to 2.0 pound force, or in a range of about 1.0 pound force to about 1.5 pound force, which is similar to the actuation force on the button to deploy the implant when there is no plug present in the bore.

The need for frequent intraocular injections in patients means the injections should be easy and convenient to administer. This goal is sometimes at odds with the need to make the injections safe and painless. The plug within the bore makes the applicator more user-friendly so that the applicator may be casually handled without fear of the implant slipping out of the bore prior to implantation. However, the plug within the bore can be too effective at preventing the implant for slipping out of the bore. The retainer solution of the plugs can be calibrated to the bore size of the cannula so that the plug that forms fully spans the bore rather than simply coating the walls to prevent the implant from inadvertently falling out of the cannula while still being sufficiently frangible that the implant can pass through the bore at the time of actuation and deployment within the eye.

HPMC has been used in combination with polystyrene microbeads to achieve higher IOP elevations in mouse models of glaucoma (Liu and Ding “Establishment of an experimental glaucoma animal model: A comparison of microbead injection with or without hydroxypropyl methylcellulose” Experimental and Therap. Med. 14:1953-1960, 2017). Pleasingly, the retention plug formed of 3% F4M-HPMC solution for retaining the Bimatoprost SR implants and Ozurdex implants did not cause any detectable adverse events when used for this purpose.

Methods of Implantation

The delivery devices described herein allow for the implant(s) to be deployed in a location in the eye that allows for multiple administrations with decreased risk of corneal adverse events, in particular, endothelial cell loss. Positioning the implant behind the iris within the posterior chamber, and preferably within the ciliary sulcus, can decrease the incidence of corneal adverse events and provides more effective drug delivery compared to intracameral placement described in more detail below. The method of implantation to position the implant can vary depending on whether the patient is phakic or pseudophakic. For example, pseudophakes may be implanted with a more conventional beveled needle tip that is sharp, whereas it is preferable, although not required, to use a cannula having a blunt tip for phakic patients to avoid inadvertent damage to the natural lens. The cannula can be designed specifically to deploy implant(s) into the posterior chamber of a patient's eye due to its blunt, atraumatic distal tip having rounded inner and outer edges and overall flexibility that allows for ab interno access to the posterior chamber and, preferably, the ciliary sulcus. Implants deployed within this location have the distinct advantage of lower risk of corneal endothelial cell disruption while providing long term reduction of intraocular pressure for a patient suffering from glaucoma.

The method of implantation may involve accessing the target area within the ocular region with a delivery apparatus 200. FIGS. 1E-1H illustrate in schematic a method of delivering an implant ab interno to the ciliary sulcus. A sharp tool having a distal end configured to penetrate the cornea may be used to create an opening in the eye and through which the blunt tipped cannula is inserted. In an implementation, a separate first tool 305, such as a hypodermic needle (approximately 35 gauge up to 28 gauge), knife-tipped device, surgical blade, or diamond knife, can be used to form a puncture or stab incision in the cornea 102 to initially enter the anterior chamber 106 (see FIG. 1E) and the cannula 240 inserted after through the opening (FIG. 1F). The opening can be approximately 1 mm wide. The opening can be a self-sealing corneal incision, for example, an incision that is about 1 mm in size and no greater than about 2.85 mm. The opening into the anterior chamber 106 can be made in the eye, such as at the level of the limbus of the cornea 102 in the superior-nasal quadrant with the aid of a surgical microscope. The corneal opening can be made superiorly or temporally. This access site provides an appropriate angle for insertion of the cannula 240 into the posterior chamber 108 on the opposite side. The implant 10 may be placed within the lower hemisphere of the eye, preferably at a 6 o'clock position. When placing the implant inferiorly, the cannula 240 can be inserted tangentially from a temporal approach toward the ciliary sulcus 111 with a widely dilated pupil 105. The cannula 240 can be inserted through the corneal opening into the anterior chamber 106 and through the pupil 105 and under at least a portion of the iris 104. The pupil 105 can be dilated with mydriatics such as tropicamide 0.5% to 1%. Phenylephrine 2.5% can be additionally used as well as other agents known to dilate the pupil including atropine, cyclopentolate, homatropine. Dilating the pupil can improve access to the ciliary sulcus region. Viscoelastic can be used to deepen the anterior chamber 106 as well as the posterior chamber 108, particularly the space between the back of the iris 104 and the front of the lens 111 to improve access as well.

The distal tip of the cannula 240 can be advanced through the anterior chamber 106 at least partially into the posterior chamber 108 (see FIG. 1G) and deploy the implant 10 using the push rod 248 from the cannula 240 within or near the ciliary sulcus 111 (see FIG. 1H). The tip of the cannula 240 can be directed about 1-2 mm past the iris fringe within the posterior chamber prior to ejection of the implant from the lumen. In other implementations, the tip of the cannula 240 can be directed deep into the posterior chamber 108 within the ciliary sulcus 111. In other implementations, the cannula 240 may be advanced sufficiently so that the implant 10 exiting the cannula 240 may also rest on or within the zonules or another location of the posterior chamber 108 behind the iris 104. The cannula 240 need not deploy the implant 10 directly into the posterior chamber 108. For example, the distal tip of the cannula 240 can be advanced to a location over the lens 110 of the eye and released onto the lens 110. Thereafter, an iris spatula or forceps, like a long-bladed Kelman McPherson's forceps, may be used to aid in manually placing the implant 10 to a desired location behind the iris 104 into the posterior chamber 108.

As discussed in detail above, the distal end of the cannula 240 can be blunt with rounded edges, particularly when implanting a device into the posterior chamber 108 of phakic patients, so as to avoid penetrating the lens capsule inadvertently as the cannula 240 is inserted through the pupil 105 and over the anterior surface of the lens 110. Once the distal opening of the cannula 240 is positioned behind the iris 104 within the posterior chamber 108 near or within, for example, the ciliary sulcus 111, the actuator 250 can be activated to deploy the implant out from the lumen 225 of the cannula 240 into the ciliary sulcus 111 of the posterior chamber 108. The actuator 250 engages a linkage 260 that can be gradually depressed as the user pushes against the actuator 250. This gradual depression allows for a user to slowly advance the push rod 248 through the lumen 225 of the cannula 240 to advance the implant 10 distally towards the distal open. The implant 10 is urged past the retention plug 280 such that the plug 280 and the implant 10 are released together within the eye.

After placement of the implant 10 and withdrawal of the cannula 240 from the eye, the pupil 105 can be brought down (constricted) with a topical or intracameral muscarinic agonist such as pilocarpine or intracameral Miochol (acetylcholamine) or Miostat (carbachol). This helps secure or “capture” the implant within the posterior chamber 108 and reduces the chance that the implant migrates in front of the iris 104 and out of the ciliary sulcus 111 region. Additionally, rotation of the eye in a slightly downward direction, or with the head positioned with the chin tilted downwards, also improves placement of the implant inferiorly in the immediate post-op period.

If the anterior chamber 106 loses aqueous or the depth is shallow, viscoelastic substances or OVDs (ophthalmic visco-surgical devices) can be used to deepen or maintain the anterior chamber depth to gain better access to the posterior chamber 108. Cohesive viscoelastics are preferred since they can be more easily removed. BSS can be injected to deepen the chamber following the corneal incision and before the cannula 240 is inserted to maintain depth of the anterior chamber 106.

Multiple implants can be placed through one cannula 240 dependent on how many implants were initially placed in the cannula 240. The viscoelastic substance can be removed from the eye at the end of the procedure using an irrigation/aspiration procedure to prevent an IOP spike, taking care not to displace the posterior chamber implant. The incisional area can be closed as necessary with sutures or using stromal hydration with BSS.

### EXPERIMENTAL

### Example 1: Dose-Response in Reduction in Intraocular Pressure (IOP) with Anterior Chamber Implant

Sixty male normotensive beagle dogs implanted with different implant sizes and bimatoprost doses were evaluated: implant length/dose of 1 mm/8 μg, 1.5 mm/15 μg, 1 mm/30 μg, and 2 mm/60 μg. Implants were administered using a 25-gauge applicator device. The right eye of animals received a single administration of implant at doses 8 μg, 15 μg, 30 μg, and 60 μg. The untreated left eye was used to evaluate within-animal differences between treated and fellow eyes. Both eyes were prepared for intracameral implant administration by applying topical ophthalmic anesthetic (1 or 2 drops of 0.5% proparacaine) approximately 5 minutes before dose administration. Eyes were rinsed with a diluted iodine solution for 2 to 3 minutes and the periorbital region cleaned with cotton-tipped applicators. The eyes were then irrigated with saline, and topical ophthalmic anesthetic instilled to each eye. The implant was placed into the anterior chamber using the sterile, preloaded applicator, and a broad-spectrum antibiotic was topically applied to the eye.

IOP measurements were taken in the treated and untreated eye. A dose-dependent IOP-lowering effect in terms of mean IOP change in the treated right eyes from baseline (corrected for untreated left eye) was evident following administration of the implant, with all dose strengths reducing IOP through at least 90 days post-dose. The mean baseline IOP values (combined mean IOP values on study days −5 and −7) for the right and left eyes, respectively, were 14.8 and 14.3 mm Hg (8 μg), 14.0 and 13.6 mm Hg (15 μg), 14.9 and 14.6 mm Hg (30 μg), and 13.0 and 12.3 mm Hg (60 μg), demonstrating comparable IOP values at baseline between the 2 eyes.

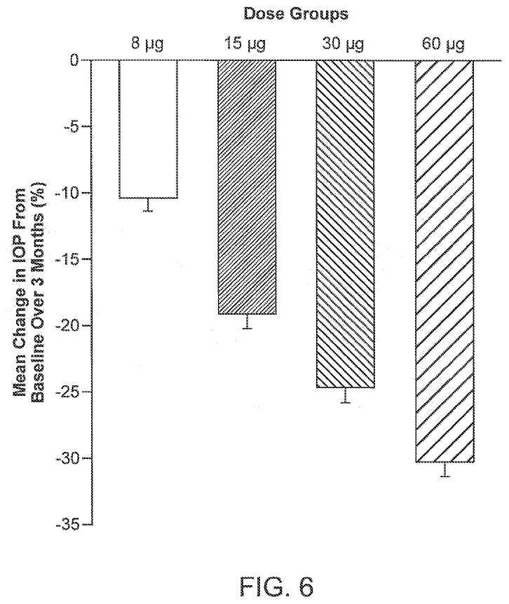

FIG. 6 shows the mean percentage change in intraocular pressure (IOP) from baseline in beagle dogs in the treated right eye over 3 months after intracameral administration of the implant. Error bars indicate the standard error of the mean. The IOP was lowered following placement of the anterior chamber implant and a clear dose response was evident. The mean percentage reduction in IOP from baseline was about 10% (8 μg), 19% (15 μg), 24% (30 μg), and 30% (60 μg).

Ophthalmic examinations were conducted using a slit-lamp bio-microscope. The Standardization of Uveitis Nomenclature scoring system was used to evaluate anterior chamber cells, anterior chamber flare, and conjunctival hyperemia. Corneal thickness (pachymetry) measurements were taken using AccuPach V (Accutome, Malvern, PA, USA). Noncontact specular microscopy (TOPCON SP-300; Topcon Corporation, Tokyo, Japan) was conducted in both eyes to assess corneal endothelium. Intracameral placement of bimatoprost implant at all doses was well tolerated. Generally, implantation-site findings were minimal and resolved by day 8 or 9 post-dose. Mild to moderate conjunctival hyperemia (+1 to +2) was evident in bimatoprost treated eyes with a dose-depending trend. Aqueous flare was sporadically and infrequently seen in all groups during the first week and resolved in all groups by day 8 or 9. There were no clear and consistent differences, or clinically meaningful abnormalities, in corneal thickness measurements between left and right eyes. Pupil diameter measurements indicated a dose-associated miosis in the bimatoprost treated eye that diminished over the course of the study. Bimatoprost implants had no effect on macroscopic or microscopic findings assessed at 4 or 6 months following implantation, other than minimal attenuation of the corneal endothelium in the bimatoprost implant at 60 μg treated eye at 4 months and another at 6 months, which was considered to be related to the implant rather than to bimatoprost.

### Example 2: Reduction in Intraocular Pressure (IOP) with Posterior Chamber Implant Compared to Anterior Chamber Implant

A patient with glaucoma was treated with a 10 ug bimatoprost (20% by weight) implant of the formulation set out in Table 1. The patient's baseline intraocular pressure was 24.5 mm Hg. The implant was initially positioned in the anterior chamber of the left eye and allowed to settle within the iridocorneal angle. IOP was measured at hour 0 in both eyes at weeks 4, 6, 8, 12, and 16 following implantation. The IOP was 17 mm Hg at week 4. The average IOP for the anterior chamber implant at weeks 4, 6, and 8 was 15 mm Hg, which was a 38.8% reduction in IOP over baseline. The implant moved from the anterior chamber to the posterior chamber between week 8 and week 12. The IOP was 15 mm Hg at week 12 and 11.5 mm Hg at week 16. The posterior chamber implant had an average IOP at weeks 12 and 16 of 13.3 mm Hg or a 45.7% reduction in IOP over baseline. The posterior chamber implant caused a greater reduction in IOP compared to the anterior chamber implant.

The patient continued to have IOP control with the posterior chamber implant until the 18 month time-point when topical glaucoma medication was initiated. Anterior chamber implants typically reduce IOP for only 9 months on average. Thus, the posterior chamber implant had a greater reduction in IOP over baseline for a prolonged period of time compared to the anterior chamber implant.

Posterior chamber placement of an implant increased the distance between the source of drug and target tissues compared to the anterior chamber placement. According to Fick's Second Law of Diffusion, 3-5 mm distance between drug source and target tissue ought to have led to a reduction of the drug concentration in the target tissues by about 5% (or from 7.5% to 12.5%). One would not have expected the posterior chamber implant, which was further away from the target tissues and lower theoretical drug concentration, to have had higher effectiveness in IOP reduction compared with the anterior chamber implant, which was positioned closer to the target tissues and higher theoretical drug concentration. One would not have expected the posterior chamber implant to also reduce IOP for a longer period of time compared to the anterior chamber implant.

### Example 3: Reduction in Intraocular Pressure (IOP) with Posterior Chamber Implant

The efficacy and ocular tolerability of posterior chamber placement of a sustained release bimatoprost implant was studied. IOP was measured in 3 normotensive beagle dogs implanted with a 30 μg bimatoprost implant in the posterior chamber. The 30 μg implant included 20% w/w bimatoprost, 45% w/w PLA (Resomer R203S), 20% w/w PLGA (Resomer RG752S), 10% w/w PLA (Resomer R02H), 5% w/w PEG-350. The implant was 2.3 mm in length and 250 μm in diameter. Implants were administered using a 25-gauge applicator device. The right eye of animals received a single administration of the implant and the contralateral eye was untreated and used to evaluate within-animal differences between treated and fellow eyes.

Dogs were dosed with a pre-anesthetic subcutaneous injection of atropine (0.022 mg/kg; Med-Pharmex Inc., Pomona, CA) and then anesthetized with an intravenous cocktail of ketamine (6.25 mg/kg; Putney, Portland, ME), xylazine (0.625 mg/kg; Akorn, Inc, Decatur, IL) and acepromazine (0.125 mg/kg; Phoenix, St. Joseph, MD). Lid and periocular areas were prepped with 5% povidone/iodine solution for 3 minutes then washed out with an irrigation of balanced salt solution (BSS). Mydriatics were instilled to open the pupil in order to provide a larger area of access for placement of an implant behind the iris. The applicator was partially inserted through the clear cornea in the superior-nasal quadrant with the aid of a surgical microscope. The implant was dosed into the anterior chamber and placed on the lens. With the aid of an iris spatula, the implant was placed behind the iris into the posterior chamber. Zymaxid® (gatifloxacin ophthalmic solution, Allergan, Irvine CA) was applied topically after the procedure.

Intraocular pressure was measured with a hand-held rebound tonometer (TonoVet; ICare, Helsinki, Finland) using the dog setting. Measurements were taken at the same time in the morning. Two measurements were taken per eye (6 values averaged for each measurement). Pupil diameter was measured with a Medimeter ruler with pupil gauge (Prestige Medical, Northridge, CA). Non-contact specular microscopy was conducted in both eyes to assess corneal endothelium and to measure central cornea endothelial cell density.

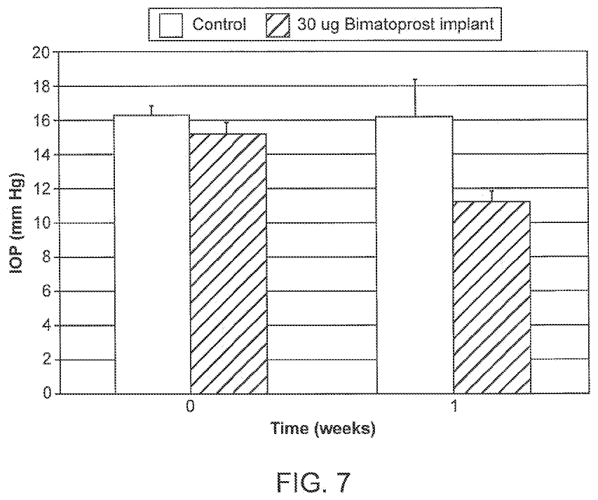

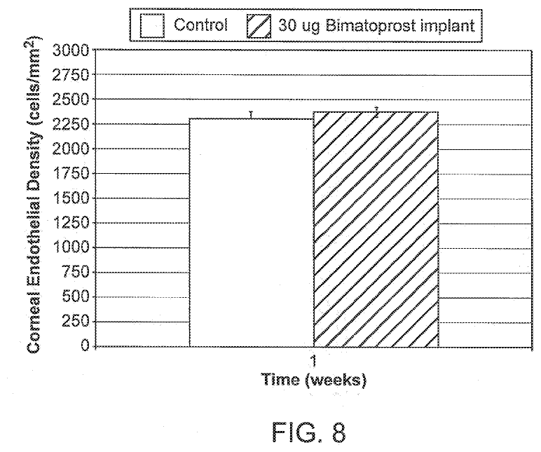

IOP measurements were taken in the treated and untreated eyes before implantation and week 1 after dosing. Mean baseline IOP were 15.2 and 16.2 mm Hg in the study and non-study eye, respectively. At 1-week post dosing, IOP decreased by 30% compared to fellow eye with the 30 μg bimatoprost implant (FIG. 7). There was no evidence of adverse events including anterior segment inflammation and no changes to endothelial cell density. FIG. 8 shows the central cornea endothelial cell density at 1 week after dosing was 2377 cells/mm² and 2299 cells/mm² in the study and non-study eye, respectively. There also was no pigment dispersion observed.

The 30 μg bimatoprost implant dosed into the anterior chamber as described above in Example 1 decreased IOP by 26% at 1-week post-dosing with no adverse events (i.e., adverse tolerability findings). The 30 μg bimatoprost implant dosed into the posterior chamber decreased IOP by 30% at week 1 post-dosing with no adverse events. IOP lowering was greater with the 30 μg bimatoprost implant positioned in the posterior chamber compared to the 30 μg bimatoprost implant positioned in the anterior chamber.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements, embodiments, or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

### P EMBODIMENTS

P Embodiment 1. A method for improving the efficacy of a prostamide-containing implant in reducing intraocular pressure, the method comprising: positioning the prostamide-containing intraocular implant into a posterior chamber of an eye of a patient in need thereof, and wherein the prostamide-containing implant causes a greater reduction in intraocular pressure of the patient in need thereof compared to the same prostamide-containing implant positioned into an anterior chamber of the eye of the patient.

P Embodiment 2. The method of P embodiment 1, wherein the patient has glaucoma or ocular hypertension.

P Embodiment 3. The method of P embodiment 1 or 2, wherein the prostamide-containing intraocular implant resides within the posterior chamber releasing the prostamide for a period that is at least about 12 weeks up to about 24 months.

P Embodiment 4. The method of any one of P embodiments 1-3, wherein the prostamide-containing intraocular implant comprises bimatoprost or a salt thereof present in an amount of about 20% by weight of the implant.

P Embodiment 5. The method of any one of P embodiments 1-4, wherein the prostamide-containing intraocular implant comprises 6 μg, 10 μg, 15 μg, or 20 μg of bimatoprost or a salt thereof.

P Embodiment 6. The method of any one of P embodiments 1-5, wherein the prostamide-containing intraocular implant comprises a biodegradable polymer matrix comprising at least one biodegradable polymer.

P Embodiment 7. The method of any one of P embodiments 1-6, wherein the prostamide-containing intraocular implant comprises a biodegradable polymer matrix, polyethylene glycol 3350, and a prostamide as the active agent, wherein the prostamide and polyethylene glycol 3350 are associated with the biodegradable polymer matrix, which comprises:
a) an ester end poly(D,L-lactide),
b) an acid end poly(D,L-lactide), and
c) an ester end poly(D,L-lactide-co-glycolide) having a D,L-lactide to glycolide molar ratio of about 75:25;
wherein the bimatoprost or a salt thereof constitutes 18 to 22% of the implant by weight, the ester end poly(D,L-lactide) constitutes 18 to 22% of the implant by weight, the acid end poly(D,L-lactide) constitutes 13.5 to 16.5% of the implant by weight, the ester end poly(D,L-lactide-co-glycolide) constitutes 35 to 45% of the implant by weight, and wherein the polyethylene glycol 3350 constitutes 3.5 to 6.5% of the implant by weight.

P Embodiment 8. The method of any one of P embodiments 1-7, wherein the prostamide-containing intraocular implant comprises a biodegradable polymer matrix, polyethylene glycol 3350, and a prostamide as the active agent, wherein the prostamide and polyethylene glycol 3350 are associated with the biodegradable polymer matrix, which comprises:
a) an ester end poly(D,L-lactide) having an inherent viscosity of 0.25-0.35 dl/g,
b) an acid end poly(D,L-lactide) having an inherent viscosity of 0.16-0.24 dl/g, and
c) an ester end poly(D,L-lactide-co-glycolide) having an inherent viscosity of 0.16-0.24 dl/g and a D,L-lactide to glycolide molar ratio of about 75:25;
wherein the bimatoprost or a salt thereof constitutes 18 to 22% of the implant by weight, the ester end poly(D,L-lactide) constitutes 18 to 22% of the implant by weight, the acid end poly(D,L-lactide) constitutes 13.5 to 16.5% of the implant by weight, the ester end poly(D,L-lactide-co-glycolide) constitutes 36 to 44% of the implant by weight, and wherein the polyethylene glycol 3350 constitutes 3.5 to 6.5% of the implant by weight, wherein the inherent viscosity of each of the poly(D,L-lactide) and poly(D,L-lactide-co-glycolide) polymers is determined for a 0.1% solution of the polymer in chloroform at 25° C.

P Embodiment 9. The method of any one of P embodiments 1-8, wherein the implant is placed in the eye using an intraocular delivery apparatus, the apparatus comprising an elongate housing and a cannula extending longitudinally from the housing, the cannula having a proximal end and a distal blunt end and having a lumen extending therethrough, the lumen having an inner diameter sufficient to receive the implant and permit translation of the implant through the lumen and into the eye.

P Embodiment 10. The method of any one of P embodiments 1-9, wherein the implant comprises a rod shape having a diameter of about 150 μm to 300 μm and a length of about 0.5 mm to 2.5 mm.

P Embodiment 11. The method of any one of P embodiments 1-10, wherein the cannula has a gauge size selected from the group consisting of 25 gauge, 26 gauge, 27 gauge, 28 gauge, 29 gauge, 30 gauge, and 32 gauge.

P Embodiment 12. The method of any one of P embodiments 1-11, wherein the distal blunt end of the cannula is inserted through the pupil of the eye and positioned near a ciliary sulcus.

P Embodiment 13. A method for reducing intraocular pressure (IOP) in a patient in need thereof, the method comprising:
positioning one or more prostamide-containing intraocular implants into a posterior chamber of an eye of a patient in need thereof, and
wherein the one or more postamide-containing intraocular implants has a greater effect on reducing IOP of the patient in need thereof over a period of time compared to the same one or more intraocular implants positioned into an anterior chamber of the patient.

P Embodiment 14. A method of treating an ocular condition in a patient in need thereof, the method comprising:
administering to the patient an intraocular implant comprising bimatoprost and a biodegradable polymer, wherein the intraocular implant is administered within the posterior chamber of at least one eye of the patient, and
wherein the intraocular implant reduces intraocular pressure of the patient to a greater extent than the same intraocular implant administered to the anterior chamber of the eye of the patient.

P Embodiment 15. A method for improving the efficacy of a prostamide-containing implant in reducing intraocular pressure (IOP), the method comprising:
implanting the prostamide-containing intraocular implant into a posterior chamber of an eye of a patient in need thereof, and
wherein the prostamide-containing implant causes a reduction in one or more adverse events in the patient when compared to implantation of the prostamide-containing implant in an anterior chamber of the patient.

P Embodiment 16. The method of P embodiment 15, wherein the one or more adverse events comprises anterior segment inflammation, corneal endothelial cell density changes, or pigment dispersion.

P Embodiment 17. A method of implanting an intraocular implant in a patient in need thereof to treat an ocular condition, the method comprising:
forming an aperture in a cornea of an eye of the patient;
passing a cannula of an applicator through the aperture, the cannula having a lumen and a distal tip;
advancing the distal tip of the cannula through the anterior chamber towards the pupil;
implanting an intraocular implant through the lumen of the cannula; and
positioning the intraocular implant within a region of the posterior chamber of the eye behind the iris.

P Embodiment 18. The method of P embodiment 17, wherein the patient is phakic.

P Embodiment 19. The method of P embodiments 17 or 18, wherein the distal tip of the cannula is blunt.

P Embodiment 20. The method of any one of P embodiments 17-19, wherein the patient is pseudophakic.

P Embodiment 21. The method of any one of P embodiments 17-20, wherein the distal tip of the cannula is sharp.

P Embodiment 22. The method of any one of P embodiments 17-21, wherein the distal tip of the cannula is between 27 gauge and 32 gauge, and wherein the cannula has a working length that is between 12 mm and 18 mm.

P Embodiment 23. The method of any one of P embodiments 17-22, wherein the aperture is formed using a needle or a surgical blade.

P Embodiment 24. The method of any one of P embodiments 17-23, wherein the aperture is formed within a lower hemisphere of the cornea.

P Embodiment 25. The method of any one of P embodiments 17-24, wherein the aperture is made superiorly or temporally.

P Embodiment 26. The method of any one of P embodiments 17-25, further comprising dilating the pupil of the eye by administering one or more mydriatics to the patient.

P Embodiment 27. The method of any one of P embodiments 17-26, wherein the one or more mydriatics is selected from the group consisting of tropicamide and phenylephrine.

P Embodiment 28. The method of any one of P embodiments 17-27, wherein the implant is positioned within the ciliary sulcus in the posterior chamber of the eye.

P Embodiment 29. The method of any one of P embodiments 17-28, wherein the implant is positioned on or within the ciliary zonules in the posterior chamber of the eye.

P Embodiment 30. The method of any one of P embodiments 17-29, further comprising constricting the pupil by administering a muscarinic agonist.

P Embodiment 31. The method of any one of P embodiments 17-30, wherein the muscarinic agonist is selected from the group consisting of pilocarpine, acetylcholamine, and cabachol.

P Embodiment 32. The method of any one of P embodiments 17-31, further comprising deepening or maintaining a depth of the anterior chamber by injecting a viscoelastic substance or balanced saline solution after forming the aperture and before inserting the cannula.

P Embodiment 33. The method of any one of P embodiments 17-32, wherein the intraocular implant is a prostamide-containing implant that causes a reduction in intraocular pressure.

P Embodiment 34. The method of any one of P embodiments 17-33, wherein the reduction in intraocular pressure is greater than when the same prostamide-containing implant is positioned into the anterior chamber of the patient.

P Embodiment 35. The method of any one of P embodiments 17-34, wherein the patient has glaucoma or ocular hypertension.

P Embodiment 36. The method of any one of P embodiments 17-35, wherein the prostamide-containing intraocular implant comprises bimatoprost or a salt thereof.

P Embodiment 37. The method of any one of P embodiments 17-36, wherein the bimatoprost is present in an amount of about 20% by weight of the implant.

P Embodiment 38. A method for improving the efficacy of an implant in reducing intraocular pressure (IOP), the method comprising:
positioning the implant into a posterior chamber of an eye of a patient in need thereof, wherein the implant causes a greater reduction in IOP of the patient in need thereof compared to the same implant positioned into an anterior chamber of the patient.

P Embodiment 39. The method of P embodiment 38, wherein the implant delivers a prostamide or a prostaglandin analog to the eye.

P Embodiment 40. The method of P embodiment 38 or 39, wherein the implant delivers a compound selected from the group consisting of bimatoprost, latanoprost, or travoprost to the eye.

P Embodiment 41. A prostamide-containing implant for use in the reduction of intraocular pressure in a patient in need thereof,
wherein the implant is configured to be positioned in a posterior chamber of an eye of the patient in need thereof, and
wherein the implant results in a greater reduction in intraocular pressure than the same implant positioned in an anterior chamber of the eye of the patient.

P Embodiment 42. The implant of P embodiments 41, wherein the prostamide-containing intraocular implant comprises bimatoprost or a salt thereof.

P Embodiment 43. The implant of P embodiment 41 or 42, wherein the prostamide-containing intraocular implant comprises bimatoprost or a salt thereof present in an amount of about 20% by weight of the implant.

P Embodiment 44. The implant of any one of P embodiments 41-43, wherein the prostamide-containing intraocular implant comprises 6 μg, 10 μg, 15 μg, or 20 μg of bimatoprost or a salt thereof.

P Embodiment 45. The implant of any one of P embodiments 41-44, wherein the prostamide-containing intraocular implant comprises a biodegradable polymer matrix, polyethylene glycol 3350, and a prostamide as the active agent, wherein the prostamide and polyethylene glycol 3350 are associated with the biodegradable polymer matrix, which comprises:
a) an ester end poly(D,L-lactide),
b) an acid end poly(D,L-lactide), and
c) an ester end poly(D,L-lactide-co-glycolide) having a D,L-lactide to glycolide molar ratio of about 75:25;
wherein the bimatoprost or a salt thereof constitutes 18 to 22% of the implant by weight, the ester end poly(D,L-lactide) constitutes 18 to 22% of the implant by weight, the acid end poly(D,L-lactide) constitutes 13.5 to 16.5% of the implant by weight, the ester end poly(D,L-lactide-co-glycolide) constitutes 35 to 45% of the implant by weight, and wherein the polyethylene glycol 3350 constitutes 3.5 to 6.5% of the implant by weight.

P Embodiment 46. The implant of any one of P embodiments 41-45, wherein the prostamide-containing intraocular implant comprises a biodegradable polymer matrix, polyethylene glycol 3350, and a prostamide as the active agent, wherein the prostamide and polyethylene glycol 3350 are associated with the biodegradable polymer matrix, which comprises:
a) an ester end poly(D,L-lactide) having an inherent viscosity of 0.25-0.35 dl/g,
b) an acid end poly(D,L-lactide) having an inherent viscosity of 0.16-0.24 dl/g, and
c) an ester end poly(D,L-lactide-co-glycolide) having an inherent viscosity of 0.16-0.24 dl/g and a D,L-lactide to glycolide molar ratio of about 75:25;
wherein the bimatoprost or a salt thereof constitutes 18 to 22% of the implant by weight, the ester end poly(D,L-lactide) constitutes 18 to 22% of the implant by weight, the acid end poly(D,L-lactide) constitutes 13.5 to 16.5% of the implant by weight, the ester end poly(D,L-lactide-co-glycolide) constitutes 36 to 44% of the implant by weight, and wherein the polyethylene glycol 3350 constitutes 3.5 to 6.5% of the implant by weight, wherein the inherent viscosity of each of the poly(D,L-lactide) and poly(D,L-lactide-co-glycolide) polymers is determined for a 0.1% solution of the polymer in chloroform at 25° C.

P Embodiment 47. An implant substantially as described herein.

P Embodiment 48. A method substantially as described herein.

What is claimed is:

1. A system for reducing intraocular pressure of a patient in need, the system comprising:
a delivery device comprising:
a housing sized to be held by an operator and comprising an actuator;
a cannula defining a lumen and having a proximal end coupled to the housing, wherein the cannula extends along a longitudinal axis from the proximal end to a distal end, wherein the distal end has rounded inner and outer edges defining a blunt, non-beveled distal opening from the lumen; and
a retention plug adhered to the cannula spanning the lumen near the distal opening; and
an intraocular implant positioned within the lumen of the cannula proximal to the retention plug.

2. The system of claim 1, wherein the cannula has an exposed working length between the proximal end and the distal end that is between about 12 mm and about 18 mm.

3. The system of claim 1, wherein the cannula has an outer dimension sized to extend through a self-sealing corneal incision or puncture.

4. The system of claim 1, wherein the cannula is no larger than about 28 gauge.

5. The system of claim 4, wherein the cannula has a wall thickness no greater than about 50 μm.

6. The system of claim 1, wherein an outside surface of the cannula is siliconized and the lumen of the cannula is substantially non-siliconized.

7. The system of claim 1, wherein the retention plug prevents inadvertent release of the implant from the lumen prior to actuation of the device.

8. The system of claim 1, wherein the retention plug is formed from a retainer solution of hydroxypropyl methylcellulose (HPMC).

9. The system of claim 8, wherein the retainer solution has a viscosity between about 6,000 cP and about 13,000 cP.

10. The system of claim 8, wherein the retainer solution has a concentration greater than about 2.5% and less than about 4%.

11. The system of claim 8, wherein the retainer solution is dispensed within the lumen of the cannula as a dispensed mass of greater than about 100 μg and less than about 300 μg.

12. The system of claim 8, wherein the retainer solution is a 3% F4M-HPMC in water having an apparent viscosity of 8,640 cP-12,760 cP and dispensed into the cannula as a dispensed mass between 125 μg-200 μg, the cannula being 28 gauge.

13. The system of claim 1, wherein the implant has a length of no more than about 3.0 mm and a maximum width of no more than about 0.5 mm.

14. The system of claim 1, wherein the intraocular implant comprises bimatoprost or a salt thereof present in an amount of about 20% by weight of the implant and a biodegradable polymer matrix comprising at least one biodegradable polymer.

15. The system of claim 1, wherein the actuator on the housing moves a push rod through the lumen of the cannula to push the implant out from the lumen via a linkage.

16. The system of claim 15, wherein the actuator is coupled to the push rod through the linkage, the push rod being movable along the longitudinal axis as the linkage is gradually flattened as the actuator is depressed.

17. The system of claim 16, wherein the push rod has a length relative to a length of the cannula sufficient for a distal end of the push rod to advance past the distal end of the cannula upon deployment of the implant using the actuator.

18. A system for reducing intraocular pressure of a patient in need, the system comprising:
a delivery device comprising:
a housing sized to be held by an operator and comprising an actuator;
a push rod linked to the actuator;
a cannula comprising a tubular wall extending along a longitudinal axis between a proximal end coupled to the housing and a distal end, the tubular wall defining a lumen sized to slidably receive the push rod,
wherein the distal end has rounded inner and outer edges defining a blunt, non-beveled distal opening into the lumen, the tubular wall between the proximal end and the distal end is about 12 mm and about 18 mm long, and wherein the tubular wall is siliconized on its external surface and the lumen is non-siliconized; and
a retention plug contained within and spanning the lumen near the distal opening, the retention plug formed from a dispensed mass of about 3% hydroxypropyl methylcellulose (HPMC) retainer solution; and
an intraocular implant positioned within the lumen of the cannula proximal to the retention plug and distal to the push rod, the implant comprising 20% by weight bimatoprost or a salt thereof and a biodegradable polymer matrix comprising at least one biodegradable polymer.

19. A method for improving the efficacy of a bimatoprost-containing intraocular implant in reducing intraocular pressure of a patient in need thereof, the method comprising:
positioning a single bimatoprost-containing intraocular implant into a posterior chamber of an eye of the patient,
wherein the single bimatoprost-containing intraocular implant causes a greater reduction in intraocular pressure compared to an equivalent bimatoprost-containing intraocular implant positioned into an anterior chamber of the eye of the patient closer to a trabecular meshwork of the eye.

20. The method of claim 19, wherein the bimatoprost-containing intraocular implant comprises 6, 10, 15, or 20 μg of bimatoprost or a salt thereof that elutes over a period of up to about 6 months, and wherein the implant is effective to reduce the intraocular pressure of the patient over a period of time between about 12 months and about 24 months.

21. The method of claim 19, further comprising:
advancing a blunt-tipped cannula having a lumen containing the implant through the anterior chamber over at least a portion of the pupil and under at least a portion of the iris;
pushing the implant through the lumen of the cannula past a retention plug attached to the cannula so as to span the lumen and out a distal opening defined by rounded inner and outer edges of the cannula; and
releasing the implant within a region of the posterior chamber of the eye behind the iris.
